(19) United States
(12) Patent Application Publication
(10) Pub. No.: US 2026/0179724 A1
(43) Pub. Date: Jun. 25, 2026

(54) METHODS FOR PROCESSING BREAST TISSUE SAMPLES

(71) Applicants: DUKE UNIVERSITY, Durham, NC (US); THE BOARD OF TRUSTEES OF THE LELAND STANFORD JUNIOR UNIVERSITY, Stanford, CA (US)

(72) Inventors: Eun-Sil HWANG, Durham, NC (US); Robert B. WEST, Stanford, CA (US)

(21) Appl. No.: 19/125,772

(22) PCT Filed: Nov. 2, 2023
(86) PCT No.: PCT/US2023/078463

§ 371 Date: Apr. 30, 2025

Related U.S. Application Data
(60) Provisional application No. 63/422,108, filed on Nov. 3, 2022.

(51) Int. Cl. G16B 40/00

(52) U.S. Cl. CPC G16B 40/00

(57) ABSTRACT

Provided herein according to some aspects is a method for processing a tissue sample from a subject, the sample comprising cells of a breast tissue site comprising or suspected of comprising ductal carcinoma in situ (DCIS), and detecting an expression level of a plurality of genes in the cells. Also provided according to some aspects is a method for generating a classifier capable of determining a risk of DCIS recurrence and/or progression. Further provided is a system for determining the risk of DCIS recurrence and/or progression in a subject in need thereof.

### CROSS-REFERENCE TO RELATED APPLICATIONS

This application claims the benefit of U.S. Provisional Patent Application Ser. No. 63/422,108, filed Nov. 3, 2022, the disclosure of which is incorporated by reference herein in its entirety.

### FEDERAL FUNDING LEGEND

This invention was made with Government support under Federal Grant nos. U2CCA233254-01 and CA185138-01 awarded by the National Institutes of Health/NCI, and Federal Grant no. BC132057 awarded by the Department of Defense. The Federal Government has certain rights to this invention.

### BACKGROUND

As nonobligate precursors of invasive disease, precancers provide a unique vantage point to study molecular pathways and evolutionary dynamics leading to the development of life-threatening cancers. Breast ductal carcinoma in situ (DCIS) is one of the most common precancers across all tissues. Current treatment of DCIS involves breast conserving surgery or mastectomy, with the goal of preventing invasive cancer. However, DCIS consists of a molecularly heterogeneous group of lesions, with highly variable risk of invasive progression. Improved understanding of which DCIS is likely to progress could better focus treatment options.

Identification of factors associated with disease progression has been studied extensively. Epidemiologic cancer progression models indicate that clinical features like age at diagnosis, tumor grade, and hormone receptor expression may have some prognostic value, but have limited ability to identify the biologic conditions that govern DCIS progression to invasive breast cancer (IBC). Previous molecular analyses of DCIS have studied either 1) cohorts of pure DCIS with known outcomes (e.g., disease-free vs recurrent), or 2) cross-sectional cohorts of DCIS with or without adjacent IBC. These approaches have tested potentially divergent assumptions: recurrence of the DCIS as IBC may arise from neoplastic cells left behind when the DCIS was removed, be related to initial field effect, or develop independently. Longitudinal cohorts provide a perspective of cancer progression over time. Analysis of DCIS adjacent to IBC assumes these preinvasive areas are good models for pure DCIS and are ancestors of the invasive cancer cells, with synchronous lesions inferring progression. To date, these studies have not produced clear evidence for a common set of events associated with invasion.

Moreover, few genomic aberrations have been identified that can differentiate DCIS from IBC and microenvironmental processes, including collagen organization, myoepithelial changes, and immune suppression, may contribute to IBC development. Presently, it remains unknown how these different molecular axes contribute to DCIS evolution.

Improved methods of analyzing DCIS tissue that may yield risk prediction for recurrence or development of IBC are needed.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein according to some aspects is a method for processing a tissue sample (e.g., biopsy) from a subject, comprising: (a) providing the sample from the subject, said sample comprising cells of a breast tissue site of interest, said site of interest comprising or suspected of comprising ductal carcinoma in situ (DCIS) (e.g., suspected based on an abnormal mammogram), wherein said cells comprise a plurality of messenger ribonucleic acid (mRNA) molecules; and (b) detecting (e.g. optically detecting) an expression level of said plurality of mRNA molecules to thereby quantify expression levels of a plurality of genes in the cells.

In some aspects, (b) comprises reverse transcribing said plurality of mRNA molecules to generate a plurality of complementary deoxyribonucleic acid (cDNA) molecules, and subsequently detecting (e.g. optically detecting) said plurality of cDNA molecules. In some aspects, the method comprises performing nucleic acid amplification (e.g., a polymerase chain reaction (PCR) or isothermal amplification) of the plurality of cDNA molecules (e.g., before the detecting).

In some aspects, detecting comprises detecting an optical signal from a probe coupled to a cDNA molecule of said plurality of cDNA molecules. In some aspects, the optical signal is a fluorescent signal.

In some aspects, the method includes processing said cells to access (and optionally extract) the plurality of mRNA molecules prior to said detecting.

In some aspects, the sample comprises a heterogeneous mixture of cells (e.g., mixed epithelial and stromal cells) (e.g., from a core biopsy or lumpectomy).

In some aspects, the subject has undergone surgery for DCIS (e.g., lumpectomy). In some aspects, the subject has not undergone surgery for DCIS.

In some aspects, the plurality of genes comprises at least 5, 10, 15, 20, 30, 40, 50, 60, 70, 80, 90 or 100 of the genes listed in Table 1. In some aspects, the plurality of genes comprises at least 30, 50, 80, 100, 200, or 300 of the genes listed in Table 1. In some aspects, the plurality of genes comprises at least 100, 300, 500, 600, 700, or 800 of the genes listed in Table 1.

In some aspects, the method includes determining an increased or decreased risk of recurrence and/or progression of DCIS based upon the expression levels of the plurality of genes.

In some aspects, the method includes treating the subject upon determining an increased risk of recurrence and/or progression of DCIS. In some aspects, the treating comprises surgery, radiation, and/or chemotherapy (e.g., endocrine therapy).

Also provided is the use of surgery, radiation, and/or chemotherapy (e.g., endocrine therapy) in a method for treating a subject upon determining an increased risk of recurrence and/or progression of DCIS. Further provided is the manufacture of a medicament (such as chemotherapy) for use in treating a subject upon determining an increased risk of recurrence and/or progression of DCIS.

Also provided according to some aspects is a method for generating a classifier, comprising: (a) providing tissue samples (e.g., biopsies) from a plurality of subjects, said samples comprising cells of a breast tissue site of interest, said site of interest comprising or suspected of comprising ductal carcinoma in situ (DCIS) (e.g., suspected based on an abnormal mammogram), wherein said cells comprises a plurality of messenger ribonucleic acid (mRNA) molecules; (b) detecting (e.g. optically detecting) an expression level of said plurality of mRNA molecules to thereby quantify expression levels of a plurality of genes in the cells; and (c) using the expression levels of the plurality of genes to train a classifier, said classifier capable of determining a risk of DCIS recurrence and/or progression, to thereby generate the classifier.

In some aspects, (b) comprises reverse transcribing said plurality of mRNA molecules to generate a plurality of complementary deoxyribonucleic acid (cDNA) molecules, and subsequently detecting (e.g. optically detecting) said plurality of cDNA molecules. In some aspects, the method comprises performing nucleic acid amplification (e.g., polymerase chain reaction (PCR) or isothermal amplification) of the plurality of cDNA molecules (e.g., before the detecting).

In some aspects, detecting comprises detecting an optical signal from a probe coupled to a cDNA molecule of said plurality of cDNA molecules. In some aspects, the optical signal is a fluorescent signal.

In some aspects, the method includes processing said cells to access (and optionally extract) the plurality of mRNA molecules prior to said detecting.

In some aspects, the sample comprises a heterogeneous mixture of cells (e.g., mixed epithelial and stromal cells) (e.g., from a core biopsy or lumpectomy).

In some aspects, the subject has undergone surgery for DCIS (e.g., lumpectomy). In some aspects, the subject has not undergone surgery for DCIS.

In some aspects, the classifier is agnostic to the biological type of DCIS and/or subsequent invasive cancer.

In some aspects, the classifier is trained based on a subsequent ipsilateral occurrence of DCIS and/or invasive breast cancer in the plurality of subjects (e.g., within about 3, 5 or 8 years from collection of the tissue samples).

Further provided is a system for determining the risk of DCIS recurrence and/or progression in a subject in need thereof, comprising: at least one processor; a sample input circuit configured to receive a tissue sample from the subject; a sample analysis circuit coupled to the at least one processor and configured to determine gene expression levels of the tissue sample; an input/output circuit coupled to the at least one processor; a storage circuit coupled to the at least one processor and configured to store data, parameters, and/or a classifier; and a memory coupled to the processor and comprising computer readable program code embodied in the memory that when executed by the at least one processor causes the at least one processor to perform operations comprising: controlling/performing measurement via the sample analysis circuit of gene expression levels of a plurality of genes in said tissue sample; optionally, normalizing the gene expression levels to generate normalized gene expression values; retrieving from the storage circuit a DCIS classifier; entering the gene expression values into the DCIS classifier; and determining a score or risk of DCIS recurrence and/or progression based upon said DCIS classifier.

In some aspects, the plurality of genes comprises at least 5, 10, 15, 20, 30, 40, 50, 60, 70, 80, 90 or 100 of the genes listed in Table 1.

In some aspects, the plurality of genes comprises at least 30, 50, 80, 100, 200, or 300 of the genes listed in Table 1.

In some aspects, the plurality of genes comprises at least 100, 300, 500, 600, 700, or 800 of the genes listed in Table 1.

In some aspects, the classifier was generated by a method as taught herein.

### BRIEF DESCRIPTION OF THE DRAWINGS

The accompanying Figures are provided by way of illustration and not by way of limitation. The foregoing aspects and other features of the disclosure are explained in the following description, taken in connection with the accompanying example figures (“FIG.”) relating to one or more embodiments, in which:

FIG. 1 is an exemplary flow diagram illustrating cohorts and methods used in a tissue analysis described herein. Two retrospective study cohorts were generated, consisting of ductal carcinoma in situ (DCIS) patients with either a subsequent ipsilateral breast event (iBE) or no later events after surgical treatment. Translational Breast Cancer Research Consortium (TBCRC) samples were macrodissected for downstream RNA and DNA analyses. Resource of Archival Breast Tissue (RAHBT) samples were 1) macrodissected like TBCRC, or 2) organized into a tissue microarray (TMA) from which serial sections were made for RNA, DNA, and protein (MIBI) analysis (RAHBT LCM cohort). TMA cores were laser capture microdissected to ensure pure epithelial and stromal components.

FIGS. 2A-2F present validation data of the 812 gene classifier. FIG. 2A: ROC curve of the 812 gene classifier in RAHBT. FIG. 2B: Kaplan-Meier plot of time to iBE (5-year outcome) stratified by classifier risk groups in RAHBT. FIGS. 2C and 2D: Kaplan-Meier plot of time to invasive progression (full follow-up) stratified by classifier risk groups in TBCRC (FIG. 2C) and RAHBT (FIG. 2D). FIGS. 2E and 2F: Forest plot of multivariable Cox regression analysis including classifier risk groups, treatment, age, DCIS grade, and ER status for invasive iBEs (full follow-up) in TBCRC (FIG. 2E) and RAHBT (FIG. 2F).

FIGS. 3A-3B show outcome-associated pathways in individual samples. FIG. 3A: Percentage of samples in 5-year outcome groups enriched for each pathway. FIG. 3B: Plot of Pearson's correlations between pathways. Color intensity and circle size are proportional to correlation coefficients, with positive correlation indicated as “+” and negative correlation indicated as “−”.

FIG. 4 is an exemplary block diagram of a tissue processing system and/or computer program product that may be used in a platform in accordance with the present invention. A tissue processing system and/or computer program product 1100 may include a processor subsystem 1140, including one or more Central Processing Units (CPU) on which one or more operating systems and/or one or more applications run. While one processor 1140 is shown, it will be understood that multiple processors 1140 may be present, which may be either electrically interconnected or separate. Processor(s) 1140 are configured to execute computer program code from memory devices, such as memory 1150, to perform at least some of the operations and methods described herein. The storage circuit 1170 may store databases which provide access to the data/parameters/classifier used by the tissue processing system 1110 such as the list of genes, weights, thresholds, etc. An input/output circuit 1160 may include displays and/or user input devices, such as keyboards, touch screens and/or pointing devices. Devices attached to the input/output circuit 1160 may be used to provide information to the processor 1140 by a user of the tissue processing system 1100. Devices attached to the input/output circuit 1160 may include networking or communication controllers, input devices (keyboard, a mouse, touch screen, etc.) and output devices (printer or display). An optional update circuit 1180 may be included as an interface for providing updates to the tissue processing system 1100 such as updates to the code executed by the processor 1140 that are stored in the memory 1150 and/or the storage circuit 1170. Updates provided via the update circuit 1180 may also include updates to portions of the storage circuit 1170 related to a database and/or other data storage format which maintains information for the tissue processing system 1100, such as the list of genes, weights, thresholds, etc. The sample input circuit 1110 provides an interface for the tissue processing system 1100 to receive tissue samples to be analyzed. The sample processing circuit 1120 may further process the tissue sample within the tissue processing system 1100 so as to prepare the tissue sample for automated analysis.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be slightly above or slightly below (e.g., by 2%, 5%, 10% or 15%) the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

Provided herein according to embodiments are methods for processing a tissue sample from a subject. In some embodiments, the tissue sample is a breast tissue sample. In some embodiments, the sample is a biopsy (e.g., a core biopsy). In some embodiments, the tissue sample is breast tissue removed during surgery such as a lumpectomy procedure or a mastectomy procedure. In other embodiments, the sample is not obtained from surgery. The tissue sample may include cells from a site of interest, for example, a site confirmed or suspected of having a tumor or pre-cancerous cells (such as DCIS). The site of interest may, for example, be suspected of having DCIS or other pre-cancerous cells based on imaging, such as the result of an abnormal mammogram finding.

In some embodiments, the tissue sample comprises a heterogeneous mixture of cells (e.g., mixed epithelial and stromal breast tissue cells). In some embodiments, the sample contains isolated cell types, or is enriched for a particular cell type or types. Isolation of cells may be performed by any suitable method, for example, by laser-capture microdissection (LCM).

The cells of a site of interest have a plurality of messenger ribonucleic acid (mRNA) molecules reflecting expression of genes in the cells. In embodiments of the present invention, a plurality of the mRNA molecules are detected (e.g., optically detected) in order to identify and/or quantify expression levels of their corresponding genes. In some embodiments, the cells are processed (e.g., lysed and optionally mRNA molecules separated from other cell components) to access the plurality of mRNA molecules from the cells.

In some embodiments, the plurality of mRNA molecules are reverse transcribed to generate a plurality of complementary deoxyribonucleic acid (cDNA) molecules representative of the mRNA molecules, and the detection includes detecting the plurality of cDNA molecules. In some embodiments, the method includes performing nucleic acid amplification of the plurality of cDNA molecules (e.g., by polymerase chain reaction (PCR)) prior to the detection. A non-limiting example method for cDNA library preparation from mRNA molecules is Smart-3SEQ. See Foley et al., “Gene expression profiling of single cells from archival tissue with laser-capture microdissection and Smart-3SEQ,” Genome Research 29:1816-1825 (2019).

Detection may be performed by suitable means known in the art. In some embodiments, optically detecting comprises detecting an optical signal from a probe coupled to the mRNA and/or cDNA molecules. In some embodiments, the optical signal is a fluorescent signal.

The expression levels of a plurality of genes as taught herein may be informative of a biological state (e.g., DCIS), and/or prognosis of recurrence or progression of the biological state (e.g., recurrence of DCIS and/or progression to invasive breast cancer). This biological state may be considered in determining treatment options for the subject. In some embodiments, methods include determining an increased or decreased risk of recurrence and/or progression of DCIS based upon the expression levels of the plurality of genes, and may further include treating the subject upon determining an increased risk of recurrence and/or progression of DCIS. The expression levels of the plurality of genes may be determined as taught herein, e.g., by quantifying and/or detecting mRNA/cDNA molecules.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the treating comprises surgery, radiation, and/or chemotherapy (e.g., endocrine therapy).

The term “effective amount” or “therapeutically effective amount” refers to an amount sufficient to effect a beneficial or desirable biological and/or clinical result.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like, for research and/or veterinary purposes.

In some embodiments, expression levels of the plurality genes may be incorporated into a classifier. The term “classifier” refers to an analysis that uses the gene expression levels, and optionally a pre-determined coefficient (or weight) for each gene expression level component, to generate an output or score for the purpose of assignment to a category or predicted outcome. A classifier may be obtained by a procedure known as “training,” which makes use of a set of data containing observations with known category membership (e.g., recurrence or iBE after an initial finding of DCIS). Training may seek to find the optimal coefficient (i.e., weight) for each component of a set of gene expression level components, as well as an optimal list of gene expression level components to include, where the optimal result is determined by the highest achievable classification accuracy. See, e.g., U.S. Publication No. 2023/0212699.

In some embodiments, a classifier as taught herein is trained base on a subsequent ipsilateral occurrence of DCIS and/or invasive breast cancer in the plurality of subjects (e.g., within about 3, 5 or 8 years from collection of the tissue samples).

The classifier may be linear and/or probabilistic. A classifier is linear if scores are a function of summed signature values weighted by a set of coefficients. Furthermore, a classifier is probabilistic if the function of signature values generates a probability, a value between 0 and 1.0 (or between 0 and 100%) quantifying the likelihood that a subject or observation belongs to a particular category or will have a particular outcome, respectively. Probit regression and logistic regression are examples of probabilistic linear classifiers that use probit and logistic link functions, respectively, to generate a probability.

In some embodiments, the classifier/classification is “agnostic” in that it is indicative of a general biological state (e.g., risk of DCIS recurrence and/or progression), but it does not provide an indication of a particular biological pathway as a cause of the state.

In some embodiments, a method for generating a classifier as taught herein may include: (a) providing tissue samples (e.g., biopsies) from a plurality of subjects, said samples comprising cells of a breast tissue site of interest, said site of interest comprising or suspected of comprising ductal carcinoma in situ (DCIS) (e.g., suspected based on an abnormal mammogram), wherein said cells comprises a plurality of messenger ribonucleic acid (mRNA) molecules; (b) detecting (e.g. optically detecting) an expression level of said plurality of mRNA molecules to thereby quantify expression levels of a plurality of genes in the cells; and (c) using the expression levels of the plurality of genes to train a classifier, said classifier capable of determining a risk of DCIS recurrence and/or progression, to thereby generate the classifier.

In some embodiments, the generating comprises, consists of, or consists essentially of, iteratively: (i) assigning a weight for each gene expression value, entering the weight and expression value for each gene into a classifier equation and determining a score or classification for a particular outcome for each of the plurality of subjects, then (ii) determining the accuracy of classification for each outcome across the plurality of subjects, and then (iii) adjusting the weight until accuracy of classification is optimized, wherein genes having a non-zero weight are included in the Optionally, components of the classifier (e.g., genes, weights and/or classification threshold value) may be uploaded into one or more databases for later retrieval or use.

In some embodiments, the classifier is trained based on a subsequent ipsilateral occurrence of DCIS and/or invasive breast cancer in a subject as a classification (e.g., within about 3, 5 or 8 years from collection of the tissue samples).

In some embodiments, the plurality of genes may include at least 5, 10, 15, 20, 30, 40, 50, 60, 70, 80, 90 or 100 of the genes listed in Table 1, which genes were found to be differentially expressed in DCIS tissue based on an outcome, as further described in the examples provided below. In some embodiments, the plurality of genes includes at least 30, 50, 80, 100, 200, or 300 of the genes listed in Table 1. In some embodiments, the plurality of genes includes at least 100, 300, 500, 600, 700, or 800 of the genes listed in Table 1.

TABLE 1
812 Differentially Expressed Genes

| | | log2Fold | | | | | |
|---|---|---|---|---|---|---|---|
| Gene | base Mean | Change | IfcSE | stat | P-value | FDR | Compartment |
| FRS2 | 166.7259 | −0.9103 | 0.1562 | 5.8289 | 0.0000 | 0.0001 | Epithelial |
| SLC30A2 | 9.5025 | −1.3093 | 0.2365 | 5.5371 | 0.0000 | 0.0002 | NA |
| ARPC4 | 115.8311 | 0.3632 | 0.0660 | −5.5070 | 0.0000 | 0.0002 | Stromal |
| RPS10 | 186.6479 | 0.4468 | 0.0795 | −5.6194 | 0.0000 | 0.0002 | NA |
| POLE3 | 103.3779 | 0.2992 | 0.0547 | −5.4733 | 0.0000 | 0.0002 | Epithelial |
| ZYG11B | 153.6384 | −0.2592 | 0.0487 | 5.3191 | 0.0000 | 0.0002 | Stromal |
| LEPR | 81.7666 | −0.7825 | 0.1457 | 5.3701 | 0.0000 | 0.0002 | Stromal |
| SULT1C2P1 | 2.9400 | 2.8289 | 0.5364 | −5.2740 | 0.0000 | 0.0002 | NA |
| SCN7A | 15.5456 | −0.8815 | 0.1678 | 5.2521 | 0.0000 | 0.0002 | Stromal |
| SDCBPP2 | 9.6769 | −1.5592 | 0.2964 | 5.2599 | 0.0000 | 0.0002 | NA |
| MRPL45 | 52.6197 | 0.8739 | 0.1636 | −5.3418 | 0.0000 | 0.0002 | Epithelial |
| MTCO1P40 | 73.0610 | 0.7268 | 0.1380 | −5.2654 | 0.0000 | 0.0002 | Epithelial |
| MT-CO2 | 611.4592 | 0.8407 | 0.1593 | −5.2771 | 0.0000 | 0.0002 | Epithelial |
| TXN | 324.1005 | 0.3545 | 0.0678 | −5.2266 | 0.0000 | 0.0002 | Epithelial |
| AP002360.2 | 9.1223 | 0.6803 | 0.1310 | −5.1918 | 0.0000 | 0.0003 | NA |
| PPP1R14B-AS1 | 7.2024 | 1.1226 | 0.2171 | −5.1719 | 0.0000 | 0.0003 | Epithelial |
| NAA10 | 62.5264 | 0.3525 | 0.0682 | −5.1652 | 0.0000 | 0.0003 | NA |
| STUM | 10.8517 | −1.2684 | 0.2487 | 5.1001 | 0.0000 | 0.0003 | NA |
| BRK1 | 160.7369 | 0.3812 | 0.0746 | −5.1116 | 0.0000 | 0.0003 | NA |
| TPH1 | 10.7104 | −1.2124 | 0.2374 | 5.1069 | 0.0000 | 0.0003 | NA |
| RPL19 | 1759.1388 | 0.8692 | 0.1710 | −5.0833 | 0.0000 | 0.0003 | Epithelial |
| RPL24 | 576.3087 | 0.3157 | 0.0624 | −5.0620 | 0.0000 | 0.0004 | Stromal |
| PTMA | 476.3741 | 0.5529 | 0.1100 | −5.0277 | 0.0000 | 0.0004 | NA |
| EIF3K | 221.1603 | 0.2932 | 0.0583 | −5.0279 | 0.0000 | 0.0004 | NA |
| RPS2 | 311.2376 | 0.4876 | 0.0971 | −5.0191 | 0.0000 | 0.0004 | Epithelial |
| S100A11 | 614.9017 | 0.4828 | 0.0966 | −4.9995 | 0.0000 | 0.0004 | Epithelial |
| MT-ATP6 | 3469.4586 | 0.5933 | 0.1197 | −4.9581 | 0.0000 | 0.0005 | Epithelial |
| FMO4 | 11.5632 | 0.9049 | 0.1831 | −4.9426 | 0.0000 | 0.0005 | NA |
| VDAC1 | 80.7150 | 0.4241 | 0.0860 | −4.9294 | 0.0000 | 0.0006 | Epithelial |
| CYP4Z1 | 45.0136 | 1.8857 | 0.3836 | −4.9160 | 0.0000 | 0.0006 | Epithelial |
| HOXC4 | 36.0919 | −0.5941 | 0.1209 | 4.9149 | 0.0000 | 0.0006 | NA |
| SET | 220.6596 | 0.3299 | 0.0674 | −4.8920 | 0.0000 | 0.0006 | Epithelial |
| LINC02611 | 18.2469 | 1.3154 | 0.2697 | −4.8766 | 0.0000 | 0.0006 | NA |
| COX5A | 43.4693 | 0.5103 | 0.1048 | −4.8688 | 0.0000 | 0.0006 | NA |
| RPS19 | 463.8981 | 0.5656 | 0.1163 | −4.8639 | 0.0000 | 0.0006 | Stromal |
| SNORA35B | 14.1749 | −0.8234 | 0.1692 | 4.8674 | 0.0000 | 0.0006 | Stromal |
| GALNT5 | 48.9741 | −1.1435 | 0.2362 | 4.8409 | 0.0000 | 0.0007 | Epithelial |
| RN7SL151P | 16.7452 | −0.7361 | 0.1529 | 4.8142 | 0.0000 | 0.0008 | NA |
| STK38L | 53.9741 | 0.4852 | 0.1010 | 4.8027 | 0.0000 | 0.0008 | Stromal |
| TFPI2 | 58.0207 | −1.5729 | 0.3282 | 4.7926 | 0.0000 | 0.0008 | Epithelial |
| DPAGT1 | 48.0511 | 0.3700 | 0.0775 | −4.7731 | 0.0000 | 0.0009 | Epithelial |
| TDRD12 | 4.8818 | −2.0909 | 0.4385 | 4.7678 | 0.0000 | 0.0009 | NA |
| RPS7 | 72.5643 | 0.6730 | 0.1420 | −4.7386 | 0.0000 | 0.0009 | NA |
| FANCM | 28.3416 | −0.4328 | 0.0912 | 4.7444 | 0.0000 | 0.0009 | NA |
| TK1 | 42.7004 | 0.7441 | 0.1568 | −4.7457 | 0.0000 | 0.0009 | Epithelial |
| UBE3AP2 | 9.0407 | −1.2024 | 0.2538 | 4.7383 | 0.0000 | 0.0009 | Stromal |
| GLYATL2 | 40.2562 | 1.9428 | 0.4118 | −4.7178 | 0.0000 | 0.0010 | Epithelial |
| RPL3 | 112.8046 | 0.7246 | 0.1546 | −4.6863 | 0.0000 | 0.0011 | NA |
| SMDT1 | 71.6589 | 0.3735 | 0.0796 | −4.6893 | 0.0000 | 0.0011 | Epithelial |
| RPS27 | 406.7587 | 0.6804 | 0.1456 | −4.6726 | 0.0000 | 0.0012 | NA |
| RPL13A | 583.5606 | 0.4922 | 0.1055 | −4.6643 | 0.0000 | 0.0012 | NA |
| NUTF2 | 80.6522 | 0.4020 | 0.0866 | −4.6403 | 0.0000 | 0.0013 | Epithelial |
| HDGF | 537.1875 | 0.2749 | 0.0594 | −4.6248 | 0.0000 | 0.0014 | Epithelial |
| ALG10B | 30.6788 | −0.4327 | 0.0936 | 4.6229 | 0.0000 | 0.0014 | NA |
| CHGA | 4.2989 | −2.4899 | 0.5401 | 4.6099 | 0.0000 | 0.0014 | NA |
| TAGLN2 | 667.8300 | 0.3986 | 0.0866 | −4.6038 | 0.0000 | 0.0015 | Epithelial |
| RPL7A | 418.3559 | 0.4178 | 0.0910 | −4.5933 | 0.0000 | 0.0015 | NA |
| RPL18 | 1325.6953 | 0.2930 | 0.0638 | −4.5955 | 0.0000 | 0.0015 | NA |
| ENO1 | 584.1607 | 0.4004 | 0.0874 | −4.5810 | 0.0000 | 0.0015 | Epithelial |
| S100A7 | 317.6505 | 2.5077 | 0.5472 | −4.5824 | 0.0000 | 0.0015 | Epithelial |
| LIFR | 114.6394 | −0.4420 | 0.0964 | 4.5846 | 0.0000 | 0.0015 | Stromal |
| SNORA79B | 23.0955 | −0.5493 | 0.1205 | 4.5570 | 0.0000 | 0.0017 | NA |
| RPS27A | 424.8563 | 0.5321 | 0.1169 | −4.5535 | 0.0000 | 0.0017 | Stromal |
| RPL23 | 1848.7490 | 0.7410 | 0.1629 | −4.5480 | 0.0000 | 0.0017 | Epithelial |
| ATP5MG | 189.8699 | 0.3173 | 0.0699 | −4.5394 | 0.0000 | 0.0017 | NA |
| KANSL1 | 200.9551 | −0.2638 | 0.0583 | 4.5265 | 0.0000 | 0.0018 | Stromal |
| MT-CYB | 3096.6212 | 0.4939 | 0.1092 | −4.5220 | 0.0000 | 0.0018 | Epithelial |
| ST13 | 194.8926 | 0.3250 | 0.0720 | −4.5102 | 0.0000 | 0.0019 | NA |
| C1orf116 | 18.6089 | 0.7786 | 0.1735 | −4.4880 | 0.0000 | 0.0020 | Epithelial |
| PSMD7 | 161.3428 | 0.3342 | 0.0745 | −4.4893 | 0.0000 | 0.0020 | NA |
| RPL35A | 890.5315 | 0.3082 | 0.0690 | −4.4694 | 0.0000 | 0.0022 | NA |
| TTC28 | 51.4364 | −0.3625 | 0.0812 | 4.4662 | 0.0000 | 0.0022 | Stromal |
| DNPH1 | 50.7834 | 0.4114 | 0.0924 | −4.4547 | 0.0000 | 0.0022 | Epithelial |
| RBM20 | 27.8790 | −1.6059 | 0.3601 | 4.4590 | 0.0000 | 0.0022 | Epithelial |
| RPL4 | 161.8864 | 0.3800 | 0.0853 | −4.4538 | 0.0000 | 0.0022 | Stromal |
| ABCC13 | 4.0090 | −1.7983 | 0.4039 | 4.4528 | 0.0000 | 0.0022 | Epithelial |
| TOMM40 | 46.6127 | 0.3369 | 0.0757 | −4.4483 | 0.0000 | 0.0022 | Epithelial |
| NDUFB11 | 99.0407 | 0.2781 | 0.0626 | −4.4455 | 0.0000 | 0.0022 | NA |
| PGK1 | 273.8511 | 0.3304 | 0.0744 | −4.4433 | 0.0000 | 0.0022 | Epithelial |
| TNRC6A | 328.2238 | −0.2043 | 0.0461 | 4.4337 | 0.0000 | 0.0023 | NA |
| RPL18A | 28.9285 | 0.8002 | 0.1808 | −4.4252 | 0.0000 | 0.0023 | NA |
| NDUFS5 | 145.9924 | 0.3530 | 0.0799 | −4.4203 | 0.0000 | 0.0024 | Epithelial |
| JPT1 | 126.1400 | 0.5764 | 0.1307 | −4.4096 | 0.0000 | 0.0025 | Epithelial |
| IGSF10 | 28.8993 | −0.6280 | 0.1426 | 4.4040 | 0.0000 | 0.0025 | Stromal |
| PHB2 | 183.9273 | 0.3159 | 0.0717 | −4.4032 | 0.0000 | 0.0025 | Epithelial |
| PLP1 | 7.8462 | −0.9068 | 0.2061 | 4.3994 | 0.0000 | 0.0025 | Stromal |
| CPLANE1 | 193.1142 | −0.3243 | 0.0738 | 4.3946 | 0.0000 | 0.0025 | Epithelial |
| FO393411.1 | 10.9132 | 0.8037 | 0.1833 | −4.3850 | 0.0000 | 0.0026 | NA |
| RPL32 | 1407.4865 | 0.2923 | 0.0667 | −4.3813 | 0.0000 | 0.0026 | Stromal |
| COX4I1 | 269.2329 | 0.3598 | 0.0825 | −4.3584 | 0.0000 | 0.0029 | NA |
| NCL | 877.1905 | 0.2058 | 0.0473 | −4.3545 | 0.0000 | 0.0029 | NA |
| KYNU | 37.1913 | 0.9441 | 0.2174 | −4.3427 | 0.0000 | 0.0029 | NA |
| MRPL51 | 124.4353 | 0.3170 | 0.0729 | −4.3487 | 0.0000 | 0.0029 | Epithelial |
| GAPDH | 1354.1132 | 0.3685 | 0.0849 | −4.3406 | 0.0000 | 0.0029 | Epithelial |
| LIPG | 5.1049 | 0.9250 | 0.2131 | −4.3398 | 0.0000 | 0.0029 | Stromal |
| SCGB1B2P | 9.5136 | −1.6117 | 0.3710 | 4.3440 | 0.0000 | 0.0029 | NA |
| C19orf48 | 31.5983 | 0.4550 | 0.1049 | −4.3383 | 0.0000 | 0.0029 | Epithelial |
| MT-CO1 | 633.6585 | 0.4873 | 0.1123 | −4.3381 | 0.0000 | 0.0029 | Epithelial |
| AL078622.1 | 3.8600 | 1.4241 | 0.3285 | −4.3348 | 0.0000 | 0.0029 | NA |
| EEF1B2 | 309.8869 | 0.3042 | 0.0702 | −4.3316 | 0.0000 | 0.0029 | Stromal |
| FAU | 535.6733 | 0.3132 | 0.0725 | −4.3221 | 0.0000 | 0.0030 | Stromal |
| MYT1L | 3.0995 | −1.2573 | 0.2921 | 4.3042 | 0.0000 | 0.0032 | NA |
| SNU13 | 142.9673 | 0.2887 | 0.0670 | −4.3058 | 0.0000 | 0.0032 | NA |
| AC104984.4 | 52.8122 | −0.6241 | 0.1451 | 4.3006 | 0.0000 | 0.0032 | NA |
| RPS29 | 1416.6224 | 0.3674 | 0.0858 | −4.2838 | 0.0000 | 0.0034 | Stromal |
| RPLP1 | 2700.8877 | 0.3998 | 0.0933 | −4.2847 | 0.0000 | 0.0034 | NA |
| MTRNR2L8 | 17.5107 | −0.8186 | 0.1914 | 4.2768 | 0.0000 | 0.0035 | Epithelial |
| CNOT2 | 184.0274 | −0.2344 | 0.0551 | 4.2574 | 0.0000 | 0.0038 | NA |
| CD55 | 77.2275 | 0.4205 | 0.0989 | −4.2501 | 0.0000 | 0.0038 | NA |
| NDRG1 | 220.3773 | 0.5430 | 0.1277 | −4.2511 | 0.0000 | 0.0038 | Stromal |
| MMP1 | 2.6952 | 1.5527 | 0.3651 | −4.2533 | 0.0000 | 0.0038 | Stromal |
| RPL35 | 1143.5910 | 0.2425 | 0.0571 | −4.2452 | 0.0000 | 0.0038 | NA |
| RPS12 | 1223.2005 | 0.4056 | 0.0956 | −4.2424 | 0.0000 | 0.0039 | Stromal |
| S100P | 92.1684 | 1.3833 | 0.3266 | −4.2358 | 0.0000 | 0.0039 | Epithelial |
| ISG20 | 39.9208 | 0.5928 | 0.1400 | −4.2352 | 0.0000 | 0.0039 | Stromal |
| RPS16 | 858.0049 | 0.3284 | 0.0775 | −4.2373 | 0.0000 | 0.0039 | Stromal |
| FDPS | 148.0351 | 0.3522 | 0.0833 | −4.2264 | 0.0000 | 0.0040 | Epithelial |
| GALE | 29.8475 | 0.4863 | 0.1155 | −4.2109 | 0.0000 | 0.0042 | Epithelial |
| TOGARAM1 | 76.6170 | −0.2760 | 0.0655 | 4.2128 | 0.0000 | 0.0042 | NA |
| NBR1 | 176.1716 | −0.2829 | 0.0672 | 4.2108 | 0.0000 | 0.0042 | NA |
| HMGA1 | 157.8932 | 0.4291 | 0.1020 | −4.2065 | 0.0000 | 0.0042 | Epithelial |
| NUP107 | 78.2503 | −0.3125 | 0.0745 | 4.1958 | 0.0000 | 0.0044 | NA |
| NHLRC2 | 78.6647 | −0.2195 | 0.0525 | 4.1767 | 0.0000 | 0.0047 | NA |
| RPL38 | 1193.5428 | 0.3215 | 0.0770 | −4.1748 | 0.0000 | 0.0047 | NA |
| PI3 | 6.6804 | 1.6641 | 0.3991 | −4.1700 | 0.0000 | 0.0048 | NA |
| MT-ND2 | 3821.4932 | 0.4804 | 0.1153 | −4.1677 | 0.0000 | 0.0048 | Epithelial |
| MRPS21 | 131.4244 | 0.3357 | 0.0807 | −4.1618 | 0.0000 | 0.0049 | Epithelial |
| NONO | 288.4391 | 0.1728 | 0.0415 | −4.1594 | 0.0000 | 0.0049 | Epithelial |
| EXTL2 | 50.1677 | −0.2809 | 0.0678 | 4.1426 | 0.0000 | 0.0052 | Stromal |
| IGFBP6 | 101.2148 | −0.5104 | 0.1233 | 4.1399 | 0.0000 | 0.0052 | Stromal |
| MRPS11 | 57.5934 | 0.3343 | 0.0808 | −4.1373 | 0.0000 | 0.0052 | NA |
| ABCA10 | 53.2498 | −0.6209 | 0.1501 | 4.1366 | 0.0000 | 0.0052 | Stromal |
| SMIM26 | 23.3890 | 0.5016 | 0.1210 | −4.1436 | 0.0000 | 0.0052 | NA |
| TMEM47 | 75.6516 | −0.6470 | 0.1564 | 4.1368 | 0.0000 | 0.0052 | NA |
| SEC61B | 136.7901 | 0.3720 | 0.0900 | −4.1335 | 0.0000 | 0.0052 | NA |
| SEC13 | 114.6709 | 0.2734 | 0.0662 | −4.1293 | 0.0000 | 0.0053 | NA |
| ASS1 | 53.7406 | 0.6461 | 0.1566 | −4.1254 | 0.0000 | 0.0053 | Epithelial |
| YBX1 | 98.0247 | 0.4810 | 0.1168 | −4.1198 | 0.0000 | 0.0054 | NA |
| LTF | 314.4192 | 1.1584 | 0.2811 | −4.1206 | 0.0000 | 0.0054 | Epithelial |
| FH | 41.7578 | 0.4064 | 0.0988 | −4.1151 | 0.0000 | 0.0055 | Epithelial |
| FAR2 | 12.5825 | 0.7846 | 0.1909 | −4.1090 | 0.0000 | 0.0056 | NA |
| RPL12 | 819.5702 | 0.3993 | 0.0973 | −4.1025 | 0.0000 | 0.0057 | NA |
| SDAD1 | 90.9892 | 0.2556 | 0.0623 | −4.0997 | 0.0000 | 0.0057 | NA |
| C5orf46 | 5.6317 | 1.0519 | 0.2567 | −4.0981 | 0.0000 | 0.0057 | NA |
| NIBAN2 | 123.8899 | 0.2598 | 0.0634 | −4.0959 | 0.0000 | 0.0057 | NA |
| RPL23A | 259.6116 | 0.7049 | 0.1721 | −4.0963 | 0.0000 | 0.0057 | NA |
| RPS3 | 1510.5243 | 0.3452 | 0.0843 | −4.0931 | 0.0000 | 0.0057 | NA |
| RN7SKP104 | 19.5474 | −0.6489 | 0.1587 | 4.0877 | 0.0000 | 0.0058 | NA |
| IRAK1 | 148.9408 | 0.2995 | 0.0733 | −4.0876 | 0.0000 | 0.0058 | NA |
| MYO15B | 127.5696 | −0.4012 | 0.0983 | 4.0825 | 0.0000 | 0.0059 | Stromal |
| NOA1 | 25.2287 | 0.3125 | 0.0767 | −4.0716 | 0.0000 | 0.0061 | NA |
| RPL9 | 309.0343 | 0.3698 | 0.0909 | −4.0696 | 0.0000 | 0.0061 | Stromal |
| NDUFB3 | 44.4324 | 0.3507 | 0.0863 | −4.0622 | 0.0000 | 0.0062 | Epithelial |
| AMPH | 14.2340 | −0.8552 | 0.2104 | 4.0648 | 0.0000 | 0.0062 | NA |
| TIMM8B | 74.8903 | 0.3296 | 0.0811 | −4.0627 | 0.0000 | 0.0062 | NA |
| ADIPOR1 | 95.8011 | 0.3492 | 0.0860 | −4.0591 | 0.0000 | 0.0062 | Epithelial |
| EFNA5 | 58.5736 | 0.6491 | 0.1600 | −4.0563 | 0.0000 | 0.0063 | Epithelial |
| PSMB4 | 165.9808 | 0.2752 | 0.0679 | −4.0541 | 0.0001 | 0.0063 | Epithelial |
| RPL37A | 2411.0963 | 0.2885 | 0.0713 | −4.0469 | 0.0001 | 0.0063 | NA |
| MDM1 | 24.8060 | −0.3696 | 0.0913 | 4.0463 | 0.0001 | 0.0063 | NA |
| RN7SKP118 | 4.6725 | −0.7677 | 0.1897 | 4.0472 | 0.0001 | 0.0063 | NA |
| POLDIP3 | 34.5322 | 0.3045 | 0.0752 | −4.0468 | 0.0001 | 0.0063 | NA |
| AL354861.2 | 4.7135 | −0.5462 | 0.1351 | 4.0441 | 0.0001 | 0.0064 | NA |
| CYCS | 132.4572 | 0.2650 | 0.0656 | −4.0379 | 0.0001 | 0.0064 | Epithelial |
| CHST1 | 48.9111 | 1.1075 | 0.2744 | −4.0368 | 0.0001 | 0.0064 | Epithelial |
| COX7B | 47.5459 | 0.3598 | 0.0891 | −4.0367 | 0.0001 | 0.0064 | Epithelial |
| RPL29 | 166.0592 | 0.3969 | 0.0985 | −4.0298 | 0.0001 | 0.0066 | NA |
| ANP32B | 294.6327 | 0.2124 | 0.0527 | −4.0272 | 0.0001 | 0.0066 | Stromal |
| SFSWAP | 145.2728 | −0.2199 | 0.0547 | 4.0228 | 0.0001 | 0.0067 | NA |
| MON2 | 125.1904 | −0.2369 | 0.0590 | 4.0134 | 0.0001 | 0.0069 | NA |
| RNU1-82P | 3.1328 | −0.7760 | 0.1935 | 4.0106 | 0.0001 | 0.0070 | NA |
| PDZD7 | 3.2627 | −0.8745 | 0.2187 | 3.9976 | 0.0001 | 0.0073 | NA |
| RPS8P6 | 9.8689 | 0.3796 | 0.0950 | −3.9952 | 0.0001 | 0.0074 | Epithelial |
| S100A8 | 195.3024 | 1.3180 | 0.3304 | −3.9889 | 0.0001 | 0.0074 | Epithelial |
| DANCR | 68.1714 | 0.3599 | 0.0903 | −3.9847 | 0.0001 | 0.0074 | Epithelial |
| ZNF862 | 21.2554 | −0.3614 | 0.0907 | 3.9838 | 0.0001 | 0.0074 | NA |
| HOXC6 | 69.9432 | −0.3921 | 0.0984 | 3.9863 | 0.0001 | 0.0074 | NA |
| RPL21 | 77.7876 | 0.6168 | 0.1548 | −3.9842 | 0.0001 | 0.0074 | NA |
| RPL26 | 590.7807 | 0.4073 | 0.1022 | −3.9851 | 0.0001 | 0.0074 | Stromal |
| PSMB3 | 225.4197 | 0.6772 | 0.1699 | −3.9853 | 0.0001 | 0.0074 | Epithelial |
| TTC37 | 181.2177 | −0.2065 | 0.0519 | 3.9786 | 0.0001 | 0.0076 | NA |
| SEM1 | 53.0279 | 0.4108 | 0.1033 | −3.9754 | 0.0001 | 0.0076 | NA |
| PSMA7 | 402.3231 | 0.3117 | 0.0784 | −3.9747 | 0.0001 | 0.0076 | Epithelial |
| RNU2-64P | 27.8676 | 0.6992 | 0.1762 | −3.9677 | 0.0001 | 0.0078 | NA |
| RPL34 | 1340.3513 | 0.2856 | 0.0720 | −3.9660 | 0.0001 | 0.0078 | Stromal |
| MPST | 48.1154 | 0.3537 | 0.0893 | −3.9625 | 0.0001 | 0.0079 | Epithelial |
| AL133355.1 | 1.5128 | −2.0641 | 0.5217 | 3.9565 | 0.0001 | 0.0080 | NA |
| RPS15A | 201.8343 | 0.2544 | 0.0643 | −3.9563 | 0.0001 | 0.0080 | NA |
| SRCIN1 | 28.1996 | 0.9273 | 0.2347 | −3.9512 | 0.0001 | 0.0081 | Epithelial |
| RN7SL183P | 2.1394 | −0.8376 | 0.2121 | 3.9498 | 0.0001 | 0.0081 | NA |
| MRPL3 | 61.6986 | 0.2576 | 0.0653 | −3.9479 | 0.0001 | 0.0081 | Epithelial |
| TES | 134.8704 | 0.3230 | 0.0818 | −3.9468 | 0.0001 | 0.0081 | Stromal |
| TKT | 277.1517 | 0.3205 | 0.0813 | −3.9404 | 0.0001 | 0.0083 | Epithelial |
| NDUFB8 | 64.9274 | 0.3364 | 0.0854 | −3.9407 | 0.0001 | 0.0083 | NA |
| DLC1 | 133.0172 | −0.4050 | 0.1029 | 3.9356 | 0.0001 | 0.0084 | Stromal |
| AC124068.2 | 3.6993 | −0.8502 | 0.2161 | 3.9351 | 0.0001 | 0.0084 | NA |
| XRCC6 | 148.3686 | 0.2318 | 0.0589 | −3.9334 | 0.0001 | 0.0084 | Epithelial |
| PSMB7 | 137.8070 | 0.2182 | 0.0555 | −3.9312 | 0.0001 | 0.0084 | NA |
| RUNDC3B | 7.6424 | −0.5685 | 0.1448 | 3.9276 | 0.0001 | 0.0085 | Stromal |
| YWHAH | 189.9630 | 0.2664 | 0.0678 | −3.9286 | 0.0001 | 0.0085 | Stromal |
| LSM3 | 79.5947 | 0.2520 | 0.0642 | −3.9257 | 0.0001 | 0.0085 | NA |
| SNORA13 | 51.5161 | −0.5076 | 0.1294 | 3.9243 | 0.0001 | 0.0085 | NA |
| RPS21 | 1535.7095 | 0.3325 | 0.0848 | −3.9186 | 0.0001 | 0.0087 | Epithelial |
| GPATCH8 | 156.6037 | −0.2359 | 0.0603 | 3.9159 | 0.0001 | 0.0087 | Stromal |
| AL391056.1 | 1.5619 | −1.1678 | 0.2983 | 3.9144 | 0.0001 | 0.0087 | NA |
| HELLPAR | 31.4562 | −0.4695 | 0.1201 | 3.9111 | 0.0001 | 0.0088 | Stromal |
| HOXC5 | 2.0586 | −1.2031 | 0.3085 | 3.9004 | 0.0001 | 0.0092 | NA |
| GPAM | 65.7249 | −0.4966 | 0.1274 | 3.8977 | 0.0001 | 0.0092 | Stromal |
| IGIP | 37.2713 | −0.3202 | 0.0822 | 3.8951 | 0.0001 | 0.0093 | Stromal |
| ADH1B | 111.5543 | −0.6726 | 0.1731 | 3.8846 | 0.0001 | 0.0096 | Stromal |
| USP30 | 18.7925 | −0.3309 | 0.0853 | 3.8809 | 0.0001 | 0.0097 | Epithelial |
| TOMM22 | 57.9923 | 0.3284 | 0.0846 | −3.8804 | 0.0001 | 0.0097 | NA |
| ACTG1 | 1799.7067 | 0.2253 | 0.0581 | −3.8772 | 0.0001 | 0.0098 | NA |
| NPM1 | 137.2848 | 0.2925 | 0.0755 | −3.8742 | 0.0001 | 0.0099 | NA |
| OST4 | 232.6950 | 0.2709 | 0.0700 | −3.8685 | 0.0001 | 0.0100 | NA |
| RNU1-89P | 2.6856 | −0.7711 | 0.1993 | 3.8692 | 0.0001 | 0.0100 | NA |
| RN7SL8P | 11.2300 | −0.6568 | 0.1697 | 3.8705 | 0.0001 | 0.0100 | NA |
| MAGI2-AS3 | 66.0857 | −0.3800 | 0.0983 | 3.8658 | 0.0001 | 0.0100 | Stromal |
| ATF4 | 290.8105 | 0.3083 | 0.0798 | −3.8631 | 0.0001 | 0.0101 | NA |
| NPEPPS | 254.1017 | −0.2611 | 0.0676 | 3.8615 | 0.0001 | 0.0101 | Epithelial |
| C2orf27A_1 | 15.9979 | −0.6479 | 0.1680 | 3.8573 | 0.0001 | 0.0102 | Epithelial |
| CHCHD10 | 148.6283 | 0.3696 | 0.0958 | −3.8566 | 0.0001 | 0.0102 | Stromal |
| SSB | 174.5414 | 0.1695 | 0.0440 | −3.8535 | 0.0001 | 0.0103 | NA |
| LMLN | 66.2284 | −0.2968 | 0.0770 | 3.8545 | 0.0001 | 0.0103 | Epithelial |
| EPGN | 2.7792 | 1.4111 | 0.3665 | −3.8498 | 0.0001 | 0.0103 | NA |
| RSL24D1 | 76.0448 | 0.2682 | 0.0697 | −3.8503 | 0.0001 | 0.0103 | NA |
| DNAJC8 | 92.5259 | 0.2165 | 0.0563 | −3.8443 | 0.0001 | 0.0105 | Stromal |
| PQBP1 | 53.2964 | 0.2548 | 0.0663 | −3.8436 | 0.0001 | 0.0105 | NA |
| RFC4 | 22.6743 | 0.3599 | 0.0937 | −3.8406 | 0.0001 | 0.0106 | Epithelial |
| PGD | 59.0110 | 0.3964 | 0.1033 | −3.8388 | 0.0001 | 0.0106 | Epithelial |
| ATP5MD | 130.2745 | 0.3247 | 0.0846 | −3.8361 | 0.0001 | 0.0107 | Epithelial |
| ANKRD30B | 151.5538 | −1.1317 | 0.2954 | 3.8313 | 0.0001 | 0.0108 | Epithelial |
| TSHZ1 | 92.8341 | −0.2344 | 0.0612 | 3.8322 | 0.0001 | 0.0108 | Stromal |
| MIR320E | 3.9902 | −1.0044 | 0.2628 | 3.8219 | 0.0001 | 0.0112 | Stromal |
| PHF21A | 83.7804 | −0.2056 | 0.0538 | 3.8205 | 0.0001 | 0.0112 | NA |
| YDJC | 38.8244 | 0.3196 | 0.0837 | −3.8179 | 0.0001 | 0.0112 | Epithelial |
| TSPAN15 | 53.2259 | 0.4957 | 0.1299 | −3.8158 | 0.0001 | 0.0113 | Epithelial |
| SP1 | 150.5837 | −0.1832 | 0.0480 | 3.8132 | 0.0001 | 0.0114 | NA |
| EIF2S3 | 343.8412 | 0.1856 | 0.0487 | −3.8100 | 0.0001 | 0.0115 | Epithelial |
| RPS25 | 319.4263 | 0.3445 | 0.0905 | −3.8051 | 0.0001 | 0.0116 | Stromal |
| MPHOSPH6 | 89.7900 | 0.5828 | 0.1531 | −3.8059 | 0.0001 | 0.0116 | Epithelial |
| RPL37 | 1081.8818 | 0.3530 | 0.0928 | −3.8027 | 0.0001 | 0.0116 | NA |
| PPIB | 390.6639 | 0.2711 | 0.0713 | −3.8036 | 0.0001 | 0.0116 | Stromal |
| HSPE1 | 92.8636 | 0.3921 | 0.1032 | −3.7998 | 0.0001 | 0.0117 | Epithelial |
| CARTPT | 18.5166 | 2.6350 | 0.6939 | −3.7974 | 0.0001 | 0.0117 | Epithelial |
| SNORD124 | 19.2914 | −0.7209 | 0.1898 | 3.7977 | 0.0001 | 0.0117 | NA |
| GASK1B | 213.6539 | −0.4953 | 0.1305 | 3.7946 | 0.0001 | 0.0118 | NA |
| PFDN2 | 88.6498 | 0.3052 | 0.0805 | −3.7923 | 0.0001 | 0.0119 | NA |
| DUBR | 22.9058 | −0.4992 | 0.1318 | 3.7885 | 0.0002 | 0.0120 | NA |
| AC009283.1 | 27.8807 | 0.9488 | 0.2505 | −3.7875 | 0.0002 | 0.0120 | Epithelial |
| NDUFB7 | 162.3880 | 0.2362 | 0.0624 | −3.7860 | 0.0002 | 0.0120 | Epithelial |
| EIF4G1 | 391.1727 | 0.1791 | 0.0473 | −3.7828 | 0.0002 | 0.0121 | Epithelial |
| STK36 | 52.1085 | −0.3190 | 0.0844 | 3.7811 | 0.0002 | 0.0122 | Epithelial |
| RPS11 | 1898.7726 | 0.2667 | 0.0706 | −3.7782 | 0.0002 | 0.0123 | Stromal |
| CRTC2 | 26.9653 | 0.2960 | 0.0784 | −3.7766 | 0.0002 | 0.0123 | NA |
| RPL31 | 2163.4680 | 0.2399 | 0.0636 | −3.7690 | 0.0002 | 0.0126 | NA |
| RPS18 | 145.3164 | 0.6948 | 0.1846 | −3.7642 | 0.0002 | 0.0128 | NA |
| PEX5L | 6.0398 | −1.3348 | 0.3550 | 3.7605 | 0.0002 | 0.0130 | NA |
| ZNF331 | 52.1755 | −0.3480 | 0.0926 | 3.7595 | 0.0002 | 0.0130 | NA |
| RN7SL277P | 2.3939 | −0.7965 | 0.2120 | 3.7563 | 0.0002 | 0.0131 | NA |
| GNMT | 11.9556 | 0.9418 | 0.2510 | −3.7530 | 0.0002 | 0.0131 | Epithelial |
| MPZL3 | 13.9105 | 0.4399 | 0.1172 | −3.7539 | 0.0002 | 0.0131 | NA |
| PRPF39 | 60.4316 | −0.2757 | 0.0734 | 3.7536 | 0.0002 | 0.0131 | NA |
| MAP4K5 | 121.8216 | −0.2183 | 0.0582 | 3.7531 | 0.0002 | 0.0131 | Stromal |
| KCNS3 | 19.2770 | 0.5242 | 0.1400 | −3.7441 | 0.0002 | 0.0135 | NA |
| SNORD67 | 14.3998 | 0.4702 | 0.1257 | 3.7392 | 0.0002 | 0.0136 | NA |
| TUBA1C | 74.1684 | 0.4109 | 0.1099 | −3.7386 | 0.0002 | 0.0136 | Epithelial |
| AC103702.1 | 8.0264 | −1.0572 | 0.2826 | 3.7411 | 0.0002 | 0.0136 | NA |
| PITPNB | 70.2844 | 0.2656 | 0.0710 | −3.7391 | 0.0002 | 0.0136 | Stromal |
| RPS26 | 51.2479 | 0.5964 | 0.1596 | −3.7376 | 0.0002 | 0.0136 | NA |
| PPT2-EGFL8 | 5.7858 | −0.6341 | 0.1699 | 3.7326 | 0.0002 | 0.0137 | NA |
| NDUFA6 | 123.6170 | 0.2872 | 0.0769 | −3.7333 | 0.0002 | 0.0137 | Epithelial |
| LRRC8D | 18.6202 | 0.3653 | 0.0981 | −3.7243 | 0.0002 | 0.0139 | NA |
| RN7SL752P | 11.5498 | −0.6391 | 0.1714 | 3.7280 | 0.0002 | 0.0139 | NA |
| SNORA20 | 19.2818 | −0.5037 | 0.1352 | 3.7242 | 0.0002 | 0.0139 | NA |
| ATP5MF | 182.4768 | 0.2135 | 0.0573 | −3.7272 | 0.0002 | 0.0139 | Epithelial |
| RPLP0 | 645.0857 | 0.3616 | 0.0971 | −3.7245 | 0.0002 | 0.0139 | NA |
| FASN | 446.4476 | 0.5658 | 0.1519 | −3.7254 | 0.0002 | 0.0139 | Epithelial |
| EXOSC5 | 27.6071 | 0.3334 | 0.0895 | −3.7235 | 0.0002 | 0.0139 | Epithelial |
| C2orf50 | 7.0972 | −0.9094 | 0.2444 | 3.7210 | 0.0002 | 0.0139 | NA |
| LINC02055 | 3.0199 | −1.1593 | 0.3116 | 3.7208 | 0.0002 | 0.0139 | NA |
| RAPGEF3 | 42.6829 | −0.3575 | 0.0961 | 3.7216 | 0.0002 | 0.0139 | Stromal |
| SURF2 | 25.7062 | 0.2755 | 0.0741 | −3.7197 | 0.0002 | 0.0139 | Epithelial |
| LDHA | 81.0751 | 0.3668 | 0.0987 | −3.7179 | 0.0002 | 0.0139 | NA |
| PTRH2 | 43.8323 | 0.4839 | 0.1302 | −3.7181 | 0.0002 | 0.0139 | Epithelial |
| MTRNR2L6 | 28.1875 | −0.4320 | 0.1162 | 3.7169 | 0.0002 | 0.0139 | NA |
| BTNL9 | 59.4652 | −0.4962 | 0.1337 | 3.7110 | 0.0002 | 0.0142 | Stromal |
| RNF170 | 55.3759 | −0.3188 | 0.0860 | 3.7078 | 0.0002 | 0.0142 | NA |
| HOXC8 | 58.2411 | −0.4816 | 0.1299 | 3.7085 | 0.0002 | 0.0142 | NA |
| ANXA2 | 313.0419 | 0.3210 | 0.0865 | −3.7098 | 0.0002 | 0.0142 | NA |
| EBP | 48.5206 | 0.3283 | 0.0885 | −3.7083 | 0.0002 | 0.0142 | Epithelial |
| DYNC2H1 | 98.1934 | −0.3325 | 0.0897 | 3.7047 | 0.0002 | 0.0143 | Stromal |
| NTRK3 | 15.0842 | −0.6135 | 0.1657 | 3.7017 | 0.0002 | 0.0144 | Stromal |
| TTC1 | 101.4034 | 0.2108 | 0.0570 | −3.6996 | 0.0002 | 0.0145 | NA |
| BAG4 | 45.9235 | −0.4433 | 0.1199 | 3.6969 | 0.0002 | 0.0145 | Epithelial |
| HECTD2 | 35.1453 | −0.4308 | 0.1165 | 3.6980 | 0.0002 | 0.0145 | NA |
| MIF | 1.2749 | 1.1345 | 0.3069 | −3.6970 | 0.0002 | 0.0145 | NA |
| TRPC1 | 17.4067 | −0.4304 | 0.1165 | 3.6953 | 0.0002 | 0.0145 | Stromal |
| ABCA6 | 75.7506 | −0.4696 | 0.1273 | 3.6904 | 0.0002 | 0.0147 | Stromal |
| UBA52 | 610.1536 | 0.2446 | 0.0663 | −3.6910 | 0.0002 | 0.0147 | NA |
| LDHB | 125.0073 | 0.4971 | 0.1349 | −3.6858 | 0.0002 | 0.0149 | Stromal |
| KRR1 | 190.5768 | −0.2011 | 0.0546 | 3.6846 | 0.0002 | 0.0149 | Epithelial |
| DHX33 | 70.6983 | −0.2293 | 0.0622 | 3.6854 | 0.0002 | 0.0149 | NA |
| SLC25A5 | 90.3456 | 0.4516 | 0.1225 | −3.6864 | 0.0002 | 0.0149 | Epithelial |
| POLN | 17.3296 | −0.5721 | 0.1554 | 3.6826 | 0.0002 | 0.0149 | Epithelial |
| AC106037.1 | 1.7150 | −1.0507 | 0.2853 | 3.6833 | 0.0002 | 0.0149 | NA |
| SNORA75 | 82.6076 | −0.5721 | 0.1554 | 3.6815 | 0.0002 | 0.0149 | Epithelial |
| SMARCE1 | 67.0011 | −0.4998 | 0.1358 | 3.6793 | 0.0002 | 0.0149 | Epithelial |
| ECH1 | 123.0274 | 0.2912 | 0.0791 | −3.6800 | 0.0002 | 0.0149 | NA |
| HSPD1 | 203.5319 | 0.2725 | 0.0741 | −3.6782 | 0.0002 | 0.0149 | Epithelial |
| HMGCS1 | 90.4131 | 0.4169 | 0.1134 | −3.6753 | 0.0002 | 0.0150 | Epithelial |
| TMEM250 | 52.0267 | 0.2359 | 0.0642 | −3.6756 | 0.0002 | 0.0150 | NA |
| AC005670.3 | 28.1117 | −0.3235 | 0.0880 | 3.6761 | 0.0002 | 0.0150 | Stromal |
| RALGAPA1 | 84.4045 | −0.2491 | 0.0678 | 3.6732 | 0.0002 | 0.0151 | NA |
| GDAP1 | 46.8261 | −0.7848 | 0.2138 | 3.6716 | 0.0002 | 0.0151 | Epithelial |
| RPL23AP42 | 9.6966 | 0.7471 | 0.2036 | −3.6696 | 0.0002 | 0.0151 | NA |
| STK24 | 88.9784 | 0.2070 | 0.0564 | −3.6695 | 0.0002 | 0.0151 | Stromal |
| UBXN4 | 472.0169 | 0.1464 | 0.0400 | −3.6636 | 0.0002 | 0.0152 | Epithelial |
| MMADHC | 71.0778 | 0.2179 | 0.0594 | −3.6654 | 0.0002 | 0.0152 | NA |
| TDRD3 | 71.4517 | −0.2598 | 0.0709 | 3.6643 | 0.0002 | 0.0152 | Stromal |
| MPP2 | 11.9645 | −0.7737 | 0.2112 | 3.6638 | 0.0002 | 0.0152 | Epithelial |
| IL1RAPL1 | 1.7168 | −0.9888 | 0.2699 | 3.6641 | 0.0002 | 0.0152 | NA |
| PDAP1 | 32.8366 | 0.3397 | 0.0928 | −3.6620 | 0.0003 | 0.0153 | NA |
| PABPC1L | 50.9472 | −0.4318 | 0.1180 | 3.6592 | 0.0003 | 0.0154 | Epithelial |
| PSMB6 | 95.9690 | 0.2636 | 0.0721 | −3.6566 | 0.0003 | 0.0155 | Epithelial |
| LRRC37B | 6.5809 | 0.7331 | 0.2007 | −3.6530 | 0.0003 | 0.0157 | NA |
| MTHFD2 | 150.0399 | 0.3414 | 0.0935 | −3.6510 | 0.0003 | 0.0158 | Epithelial |
| NACA | 183.0502 | 0.2431 | 0.0666 | −3.6497 | 0.0003 | 0.0158 | NA |
| YWHAE | 316.3479 | 0.2937 | 0.0805 | −3.6487 | 0.0003 | 0.0158 | Epithelial |
| EDF1 | 383.6365 | 0.1861 | 0.0510 | −3.6474 | 0.0003 | 0.0158 | Epithelial |
| GABBR2 | 2.7327 | 1.3440 | 0.3689 | −3.6435 | 0.0003 | 0.0160 | NA |
| RN7SL832P | 8.0411 | −0.5117 | 0.1406 | 3.6400 | 0.0003 | 0.0161 | Stromal |
| RPL14 | 143.4067 | 0.2616 | 0.0719 | −3.6407 | 0.0003 | 0.0161 | NA |
| FABP4 | 169.0150 | −0.7346 | 0.2018 | 3.6409 | 0.0003 | 0.0161 | Stromal |
| BOLA3 | 26.6999 | 0.2998 | 0.0825 | −3.6340 | 0.0003 | 0.0163 | Epithelial |
| LINC00882 | 8.3753 | −0.5897 | 0.1622 | 3.6353 | 0.0003 | 0.0163 | NA |
| NSD3 | 305.9557 | −0.3824 | 0.1053 | 3.6330 | 0.0003 | 0.0163 | Epithelial |
| ZW10 | 21.5654 | 0.3048 | 0.0839 | −3.6328 | 0.0003 | 0.0163 | NA |
| RUNDC3A | 2.7419 | −1.5516 | 0.4269 | 3.6349 | 0.0003 | 0.0163 | NA |
| UBE2L3 | 75.3491 | 0.2635 | 0.0725 | −3.6359 | 0.0003 | 0.0163 | Epithelial |
| PABPC1 | 2791.5554 | 0.3458 | 0.0953 | −3.6307 | 0.0003 | 0.0163 | Epithelial |
| AVPR1A | 11.6101 | −0.5344 | 0.1472 | 3.6310 | 0.0003 | 0.0163 | Stromal |
| RPL36 | 567.5612 | 0.3812 | 0.1050 | −3.6293 | 0.0003 | 0.0163 | NA |
| CD163L1 | 14.0522 | 0.5722 | 0.1578 | −3.6263 | 0.0003 | 0.0165 | NA |
| IRGQ | 73.3257 | 0.2279 | 0.0628 | −3.6256 | 0.0003 | 0.0165 | Epithelial |
| SNHG5 | 9226.9532 | −0.4118 | 0.1137 | 3.6217 | 0.0003 | 0.0166 | Stromal |
| COMT | 81.7900 | 0.3657 | 0.1010 | −3.6218 | 0.0003 | 0.0166 | Epithelial |
| PRDX4 | 62.0714 | 0.3154 | 0.0871 | −3.6209 | 0.0003 | 0.0166 | NA |
| ATP5PO | 143.9241 | 0.2388 | 0.0660 | −3.6198 | 0.0003 | 0.0167 | NA |
| CLIC1 | 304.5624 | 0.1909 | 0.0529 | −3.6110 | 0.0003 | 0.0172 | NA |
| FADS2 | 58.2913 | 0.8232 | 0.2280 | −3.6106 | 0.0003 | 0.0172 | Epithelial |
| UFC1 | 188.4449 | 0.3014 | 0.0836 | −3.6074 | 0.0003 | 0.0173 | Epithelial |
| EZH1 | 70.9775 | −0.2599 | 0.0721 | 3.6069 | 0.0003 | 0.0173 | Stromal |
| EDN2 | 2.8718 | 1.2316 | 0.3416 | −3.6054 | 0.0003 | 0.0173 | NA |
| ABCA9 | 59.8059 | −0.4916 | 0.1363 | 3.6058 | 0.0003 | 0.0173 | Stromal |
| SOX2-OT | 8.4246 | −0.6734 | 0.1869 | 3.6022 | 0.0003 | 0.0174 | NA |
| RPS24 | 1643.4599 | 0.2675 | 0.0743 | −3.6020 | 0.0003 | 0.0174 | NA |
| UVSSA | 69.2945 | −0.3038 | 0.0844 | 3.5998 | 0.0003 | 0.0175 | Epithelial |
| RPS8 | 1418.8359 | 0.3567 | 0.0992 | −3.5947 | 0.0003 | 0.0177 | Stromal |
| ZFHX4 | 103.0384 | −0.4200 | 0.1168 | 3.5947 | 0.0003 | 0.0177 | Stromal |
| SRPRA | 126.5176 | 0.2419 | 0.0673 | −3.5931 | 0.0003 | 0.0177 | NA |
| UBALD2 | 102.4898 | 0.3290 | 0.0915 | −3.5951 | 0.0003 | 0.0177 | NA |
| BIRC5 | 33.5345 | 0.5915 | 0.1646 | −3.5939 | 0.0003 | 0.0177 | Epithelial |
| PSMG1 | 37.4765 | 0.2836 | 0.0789 | −3.5929 | 0.0003 | 0.0177 | Epithelial |
| MID1IP1 | 45.0926 | 0.3154 | 0.0877 | −3.5941 | 0.0003 | 0.0177 | NA |
| IDH2 | 176.7698 | 0.4095 | 0.1140 | −3.5919 | 0.0003 | 0.0177 | Epithelial |
| OXCT1 | 26.8535 | 0.4402 | 0.1227 | −3.5875 | 0.0003 | 0.0178 | NA |
| HYMAI | 7.7609 | −0.7333 | 0.2044 | 3.5874 | 0.0003 | 0.0178 | Stromal |
| SERF2 | 467.3476 | 0.3300 | 0.0920 | −3.5875 | 0.0003 | 0.0178 | Epithelial |
| IGBP1 | 67.5333 | 0.2407 | 0.0671 | −3.5866 | 0.0003 | 0.0178 | NA |
| DKC1 | 140.8681 | 0.2416 | 0.0674 | −3.5863 | 0.0003 | 0.0178 | Epithelial |
| SPCS2 | 14.6954 | 0.5223 | 0.1457 | −3.5852 | 0.0003 | 0.0178 | NA |
| TRPM7 | 139.2131 | −0.2014 | 0.0562 | 3.5848 | 0.0003 | 0.0178 | Stromal |
| SRD5A3 | 58.7737 | 0.4564 | 0.1274 | −3.5823 | 0.0003 | 0.0180 | Epithelial |
| EIF3D | 170.8675 | 0.2263 | 0.0632 | −3.5816 | 0.0003 | 0.0180 | NA |
| PSD3 | 115.8670 | −0.6910 | 0.1931 | 3.5773 | 0.0003 | 0.0182 | Epithelial |
| Z82217.1 | 4.2143 | −0.6607 | 0.1847 | 3.5763 | 0.0003 | 0.0182 | NA |
| SYP | 5.7808 | −0.8035 | 0.2250 | 3.5712 | 0.0004 | 0.0185 | NA |
| MRPL9 | 67.0801 | 0.2109 | 0.0591 | −3.5693 | 0.0004 | 0.0186 | NA |
| RPL15 | 1039.2036 | 0.2341 | 0.0656 | −3.5665 | 0.0004 | 0.0187 | Stromal |
| KRT8 | 125.7984 | 0.4565 | 0.1280 | −3.5666 | 0.0004 | 0.0187 | Epithelial |
| EIF5A | 77.8317 | 0.4260 | 0.1195 | −3.5650 | 0.0004 | 0.0187 | NA |
| MBD2 | 180.6362 | 0.1732 | 0.0486 | −3.5649 | 0.0004 | 0.0187 | Epithelial |
| REV3L | 184.5734 | −0.2466 | 0.0692 | 3.5640 | 0.0004 | 0.0188 | Stromal |
| ATP5MPL | 218.1185 | 0.2022 | 0.0567 | −3.5625 | 0.0004 | 0.0188 | Epithelial |
| AC022007.1 | 7.2901 | 0.5921 | 0.1665 | −3.5568 | 0.0004 | 0.0190 | Epithelial |
| RACK1 | 1781.0099 | 0.2181 | 0.0613 | −3.5559 | 0.0004 | 0.0190 | Stromal |
| AUXG0100005 | 26.8904 | −0.4485 | 0.1260 | 3.5591 | 0.0004 | 0.0190 | NA |
| 8.1 | | | | | | | |
| SAPCD2 | 34.0289 | 0.5327 | 0.1498 | −3.5572 | 0.0004 | 0.0190 | Epithelial |
| TMTC2 | 46.0243 | −0.4194 | 0.1179 | 3.5563 | 0.0004 | 0.0190 | Stromal |
| SNRPD2 | 246.3727 | 0.1962 | 0.0552 | −3.5568 | 0.0004 | 0.0190 | Epithelial |
| GALNT14 | 6.6549 | 0.8153 | 0.2293 | −3.5552 | 0.0004 | 0.0190 | Epithelial |
| MIEF1 | 57.1078 | 0.2467 | 0.0695 | −3.5502 | 0.0004 | 0.0193 | NA |
| ACTB | 216.6451 | 0.2614 | 0.0737 | −3.5481 | 0.0004 | 0.0193 | Stromal |
| SLC31A1 | 77.0154 | 0.3344 | 0.0942 | −3.5487 | 0.0004 | 0.0193 | Epithelial |
| HDC | 14.0639 | −0.8233 | 0.2320 | 3.5489 | 0.0004 | 0.0193 | Stromal |
| COX6B1 | 331.3832 | 0.2345 | 0.0661 | −3.5470 | 0.0004 | 0.0194 | Epithelial |
| URM1 | 79.3383 | 0.1939 | 0.0547 | −3.5455 | 0.0004 | 0.0194 | NA |
| E2F2 | 4.7378 | 0.6592 | 0.1860 | −3.5440 | 0.0004 | 0.0195 | Epithelial |
| POLD1 | 21.4023 | 0.3075 | 0.0868 | −3.5424 | 0.0004 | 0.0196 | NA |
| RPL30 | 579.4401 | 0.3016 | 0.0852 | −3.5414 | 0.0004 | 0.0196 | NA |
| CSTB | 190.7836 | 0.3920 | 0.1109 | −3.5351 | 0.0004 | 0.0200 | Epithelial |
| ITGB6 | 76.5047 | 0.6551 | 0.1854 | −3.5342 | 0.0004 | 0.0200 | Epithelial |
| PCAT6 | 8.0406 | 0.5929 | 0.1678 | −3.5328 | 0.0004 | 0.0201 | NA |
| CEP126 | 70.5061 | −0.4472 | 0.1267 | 3.5302 | 0.0004 | 0.0202 | Stromal |
| MT2A | 267.3290 | 0.5046 | 0.1430 | −3.5300 | 0.0004 | 0.0202 | NA |
| RPL10A | 175.1183 | 0.4176 | 0.1184 | −3.5261 | 0.0004 | 0.0204 | NA |
| UHRF1BP1L | 70.1374 | −0.2079 | 0.0590 | 3.5261 | 0.0004 | 0.0204 | NA |
| CD48 | 16.4704 | 0.7369 | 0.2090 | −3.5254 | 0.0004 | 0.0204 | Stromal |
| LAMA2 | 79.9367 | −0.3690 | 0.1047 | 3.5247 | 0.0004 | 0.0204 | Stromal |
| HSP90AA1 | 1519.0752 | 0.2877 | 0.0816 | −3.5241 | 0.0004 | 0.0204 | Epithelial |
| SIAH2-AS1 | 7.3244 | −1.0668 | 0.3032 | 3.5184 | 0.0004 | 0.0206 | Epithelial |
| RN7SL838P | 18.9337 | −0.5960 | 0.1694 | 3.5184 | 0.0004 | 0.0206 | NA |
| AC004223.2 | 4.4292 | −0.8046 | 0.2287 | 3.5187 | 0.0004 | 0.0206 | NA |
| MYL12A | 252.3594 | 0.2709 | 0.0770 | −3.5178 | 0.0004 | 0.0206 | Stromal |
| CLDN14 | 1.4793 | 1.0866 | 0.3089 | −3.5181 | 0.0004 | 0.0206 | NA |
| AL391832.2 | 7.1015 | 0.6034 | 0.1718 | −3.5117 | 0.0004 | 0.0210 | Epithelial |
| CFAP70 | 56.4245 | −0.4771 | 0.1359 | 3.5111 | 0.0004 | 0.0210 | Epithelial |
| PDGFD | 42.9744 | −0.4255 | 0.1212 | 3.5116 | 0.0004 | 0.0210 | Stromal |
| DNAH9 | 1.7871 | −1.2553 | 0.3575 | 3.5111 | 0.0004 | 0.0210 | NA |
| RPL11 | 1607.0729 | 0.1998 | 0.0570 | −3.5027 | 0.0005 | 0.0210 | Stromal |
| UQCRH | 229.0603 | 0.2736 | 0.0781 | −3.5027 | 0.0005 | 0.0210 | Epithelial |
| S100A7A | 10.1513 | 2.5175 | 0.7184 | −3.5043 | 0.0005 | 0.0210 | Epithelial |
| ILF2 | 134.8824 | 0.2307 | 0.0658 | −3.5078 | 0.0005 | 0.0210 | Epithelial |
| CAPN13 | 24.3169 | 0.7130 | 0.2036 | −3.5025 | 0.0005 | 0.0210 | Epithelial |
| CMSS1 | 48.9005 | 0.3070 | 0.0877 | −3.5027 | 0.0005 | 0.0210 | NA |
| AC109347.2 | 2.1030 | 0.8332 | 0.2378 | −3.5044 | 0.0005 | 0.0210 | NA |
| ST14 | 96.0440 | 0.3801 | 0.1084 | −3.5078 | 0.0005 | 0.0210 | Epithelial |
| CHAC1 | 4.0519 | 0.8981 | 0.2564 | −3.5032 | 0.0005 | 0.0210 | NA |
| CIAPIN1 | 19.4517 | 0.3510 | 0.1001 | −3.5067 | 0.0005 | 0.0210 | NA |
| EEF2 | 1944.0799 | 0.2050 | 0.0585 | −3.5068 | 0.0005 | 0.0210 | NA |
| PSMD8 | 244.2775 | 0.2654 | 0.0757 | −3.5054 | 0.0005 | 0.0210 | Epithelial |
| STARD7 | 97.3247 | 0.1875 | 0.0536 | −3.5010 | 0.0005 | 0.0211 | NA |
| HSD17B10 | 79.8656 | 0.2635 | 0.0753 | −3.4996 | 0.0005 | 0.0212 | Epithelial |
| WTAP | 73.9677 | 0.2258 | 0.0646 | −3.4986 | 0.0005 | 0.0212 | NA |
| AC062004.1 | 2.2544 | −1.0333 | 0.2955 | 3.4970 | 0.0005 | 0.0212 | Stromal |
| LGI1 | 2.0735 | −0.8941 | 0.2556 | 3.4973 | 0.0005 | 0.0212 | NA |
| KCNC2 | 69.7373 | −1.6374 | 0.4683 | 3.4963 | 0.0005 | 0.0212 | Epithelial |
| SNORA38B | 12.2830 | −0.6558 | 0.1876 | 3.4954 | 0.0005 | 0.0213 | Epithelial |
| EFHC1 | 58.3314 | −0.3259 | 0.0934 | 3.4888 | 0.0005 | 0.0216 | Epithelial |
| DHCR7 | 55.9065 | 0.5131 | 0.1471 | −3.4882 | 0.0005 | 0.0216 | Epithelial |
| ZNF26 | 65.1569 | −0.2758 | 0.0790 | 3.4904 | 0.0005 | 0.0216 | Epithelial |
| CES3 | 4.3031 | 1.2790 | 0.3666 | −3.4887 | 0.0005 | 0.0216 | NA |
| ZNF44 | 69.0307 | −0.3238 | 0.0928 | 3.4882 | 0.0005 | 0.0216 | NA |
| ACO2 | 98.4239 | 0.2376 | 0.0681 | −3.4902 | 0.0005 | 0.0216 | Epithelial |
| UQCRB | 257.9997 | 0.2499 | 0.0717 | −3.4863 | 0.0005 | 0.0217 | NA |
| POMP | 142.7961 | 0.2221 | 0.0638 | −3.4839 | 0.0005 | 0.0218 | Epithelial |
| ZNF518A | 149.9459 | −0.2173 | 0.0624 | 3.4827 | 0.0005 | 0.0219 | Epithelial |
| FAM89B | 104.1775 | 0.2240 | 0.0643 | −3.4812 | 0.0005 | 0.0219 | NA |
| RPS5 | 773.5341 | 0.2798 | 0.0804 | −3.4781 | 0.0005 | 0.0221 | NA |
| AC114490.3 | 8.4655 | −0.5209 | 0.1499 | 3.4740 | 0.0005 | 0.0223 | NA |
| EIF4A2 | 210.5167 | 0.2102 | 0.0605 | −3.4738 | 0.0005 | 0.0223 | NA |
| ZFYVE16 | 163.3559 | −0.1931 | 0.0556 | 3.4737 | 0.0005 | 0.0223 | Stromal |
| OLFML2A | 63.6707 | −0.3283 | 0.0945 | 3.4741 | 0.0005 | 0.0223 | Stromal |
| AC005921.4 | 7.0003 | −0.6337 | 0.1824 | 3.4735 | 0.0005 | 0.0223 | NA |
| CCT6A | 240.3929 | 0.1985 | 0.0572 | −3.4713 | 0.0005 | 0.0224 | Epithelial |
| ARF4 | 80.9065 | 0.2889 | 0.0832 | −3.4704 | 0.0005 | 0.0224 | NA |
| MINDY3 | 32.9399 | −0.2910 | 0.0839 | 3.4701 | 0.0005 | 0.0224 | NA |
| LMNB2 | 30.0392 | 0.2786 | 0.0803 | −3.4685 | 0.0005 | 0.0225 | Epithelial |
| CEP290 | 207.9039 | −0.2385 | 0.0688 | 3.4672 | 0.0005 | 0.0226 | NA |
| VAMP8 | 236.7002 | 0.2375 | 0.0685 | −3.4662 | 0.0005 | 0.0226 | Epithelial |
| TMA7 | 78.1268 | 0.2935 | 0.0847 | −3.4656 | 0.0005 | 0.0226 | NA |
| RPS20P22 | 17.0242 | −0.6370 | 0.1840 | 3.4630 | 0.0005 | 0.0227 | NA |
| RPL8 | 2501.6919 | 0.3032 | 0.0875 | −3.4634 | 0.0005 | 0.0227 | Epithelial |
| GRB14 | 33.5959 | −1.2205 | 0.3525 | 3.4620 | 0.0005 | 0.0227 | Epithelial |
| ZNF236 | 57.4095 | −0.2533 | 0.0732 | 3.4615 | 0.0005 | 0.0227 | Epithelial |
| MT-ND4 | 11168.5621 | 0.3125 | 0.0903 | −3.4605 | 0.0005 | 0.0228 | Epithelial |
| CERS5 | 51.1223 | −0.2097 | 0.0606 | 3.4594 | 0.0005 | 0.0228 | NA |
| RP9 | 16.7409 | 0.3337 | 0.0966 | −3.4551 | 0.0006 | 0.0231 | NA |
| AC004825.3 | 2.8839 | −0.8011 | 0.2318 | 3.4558 | 0.0005 | 0.0231 | Stromal |
| MED1 | 401.9248 | 0.6233 | 0.1804 | −3.4548 | 0.0006 | 0.0231 | Epithelial |
| DHX16 | 51.7999 | −0.2590 | 0.0751 | 3.4490 | 0.0006 | 0.0235 | NA |
| ITGA2B | 1.9689 | −0.8818 | 0.2557 | 3.4488 | 0.0006 | 0.0235 | NA |
| PFKFB2 | 22.8852 | 0.4161 | 0.1208 | −3.4445 | 0.0006 | 0.0236 | NA |
| MPV17L | 40.6703 | 0.7253 | 0.2105 | −3.4463 | 0.0006 | 0.0236 | Epithelial |
| AC091153.1 | 2.0645 | 0.7727 | 0.2243 | −3.4441 | 0.0006 | 0.0236 | NA |
| MIA | 18.5782 | 0.6622 | 0.1922 | −3.4445 | 0.0006 | 0.0236 | NA |
| UFD1 | 71.2209 | 0.2409 | 0.0699 | −3.4459 | 0.0006 | 0.0236 | NA |
| PIN4 | 41.6164 | 0.2638 | 0.0766 | −3.4438 | 0.0006 | 0.0236 | Epithelial |
| S100A9 | 450.5395 | 1.0411 | 0.3026 | −3.4409 | 0.0006 | 0.0238 | Epithelial |
| PCSK2 | 1.5712 | −1.2019 | 0.3495 | 3.4389 | 0.0006 | 0.0240 | NA |
| LPL | 84.5276 | −0.5796 | 0.1686 | 3.4370 | 0.0006 | 0.0240 | Stromal |
| CHP1 | 97.8210 | 0.2366 | 0.0688 | −3.4372 | 0.0006 | 0.0240 | Epithelial |
| TXNL4A | 133.6549 | 0.2374 | 0.0691 | −3.4371 | 0.0006 | 0.0240 | Epithelial |
| SCN8A | 13.5754 | −0.7602 | 0.2214 | 3.4341 | 0.0006 | 0.0241 | Epithelial |
| STXBP6 | 4.1166 | −0.7681 | 0.2236 | 3.4346 | 0.0006 | 0.0241 | NA |
| SAP30 | 17.8093 | 0.3905 | 0.1138 | −3.4324 | 0.0006 | 0.0241 | Epithelial |
| HERC1 | 213.5943 | −0.2271 | 0.0661 | 3.4325 | 0.0006 | 0.0241 | Stromal |
| MT-ND4L | 238.5799 | 0.3945 | 0.1149 | −3.4334 | 0.0006 | 0.0241 | Epithelial |
| PLA2G2D | 7.5350 | 0.9721 | 0.2833 | −3.4309 | 0.0006 | 0.0242 | NA |
| MYOZ3 | 3.7975 | −0.8959 | 0.2611 | 3.4308 | 0.0006 | 0.0242 | NA |
| BRCA1 | 37.6428 | −0.3946 | 0.1151 | 3.4295 | 0.0006 | 0.0242 | Epithelial |
| ST6GAL1 | 44.3851 | 0.5323 | 0.1552 | −3.4289 | 0.0006 | 0.0243 | Stromal |
| PRR11 | 40.9090 | 0.5558 | 0.1623 | −3.4243 | 0.0006 | 0.0246 | NA |
| EXOC4 | 124.1939 | −0.2010 | 0.0587 | 3.4232 | 0.0006 | 0.0247 | NA |
| TMSB10 | 1768.3385 | 0.3286 | 0.0960 | −3.4218 | 0.0006 | 0.0247 | NA |
| SLC35C1 | 26.7921 | 0.3887 | 0.1136 | −3.4221 | 0.0006 | 0.0247 | NA |
| RARRES1 | 101.2983 | 0.6837 | 0.1999 | −3.4200 | 0.0006 | 0.0248 | NA |
| PFN1 | 678.8930 | 0.2656 | 0.0777 | −3.4190 | 0.0006 | 0.0249 | Stromal |
| ATP1A1 | 323.9202 | 0.2442 | 0.0714 | −3.4178 | 0.0006 | 0.0249 | Epithelial |
| AP005121.1 | 9.8503 | −1.3019 | 0.3809 | 3.4181 | 0.0006 | 0.0249 | Epithelial |
| NPHP3 | 72.6266 | −0.2324 | 0.0680 | 3.4164 | 0.0006 | 0.0249 | Stromal |
| KIAA1324L | 33.3473 | −0.4499 | 0.1317 | 3.4154 | 0.0006 | 0.0249 | NA |
| AC008264.2 | 12.5942 | −0.3474 | 0.1017 | 3.4157 | 0.0006 | 0.0249 | Stromal |
| PPP1R14B | 88.0169 | 0.3390 | 0.0993 | −3.4138 | 0.0006 | 0.0250 | Epithelial |
| TNFRSF12A | 113.9624 | 0.4140 | 0.1213 | −3.4125 | 0.0006 | 0.0251 | NA |
| DCX | 16.1216 | −0.9160 | 0.2685 | 3.4114 | 0.0006 | 0.0251 | NA |
| CDC34 | 54.5584 | 0.2676 | 0.0785 | −3.4094 | 0.0007 | 0.0253 | Epithelial |
| SERBP1 | 329.0849 | 0.1647 | 0.0483 | −3.4061 | 0.0007 | 0.0254 | NA |
| OLA1 | 60.7751 | 0.3401 | 0.0998 | −3.4068 | 0.0007 | 0.0254 | Epithelial |
| CPLX2 | 1.2203 | −3.0295 | 0.8895 | 3.4059 | 0.0007 | 0.0254 | NA |
| TMEM98 | 49.7544 | −0.4023 | 0.1181 | 3.4059 | 0.0007 | 0.0254 | Stromal |
| B3GALT5 | 24.6858 | −0.8210 | 0.2411 | 3.4052 | 0.0007 | 0.0254 | Epithelial |
| RGS5 | 659.7148 | −0.5406 | 0.1590 | 3.4006 | 0.0007 | 0.0258 | NA |
| ADAMTS9-AS2 | 7.7106 | −0.6100 | 0.1794 | 3.4007 | 0.0007 | 0.0258 | Stromal |
| RPS13 | 176.9819 | 0.5086 | 0.1496 | −3.4000 | 0.0007 | 0.0258 | NA |
| RPS19BP1 | 105.0341 | 0.2244 | 0.0660 | −3.3987 | 0.0007 | 0.0258 | Epithelial |
| GK5 | 123.1691 | −0.2370 | 0.0698 | 3.3972 | 0.0007 | 0.0259 | Epithelial |
| BCAN | 1.1052 | −1.2684 | 0.3736 | 3.3955 | 0.0007 | 0.0260 | NA |
| NCOA1 | 170.0628 | −0.1788 | 0.0527 | 3.3946 | 0.0007 | 0.0261 | Stromal |
| ZNF25 | 37.6739 | −0.2588 | 0.0763 | 3.3926 | 0.0007 | 0.0262 | Stromal |
| HYOU1 | 129.8035 | 0.2874 | 0.0847 | −3.3917 | 0.0007 | 0.0262 | Epithelial |
| LAD1 | 63.1272 | 0.5913 | 0.1744 | −3.3903 | 0.0007 | 0.0263 | Epithelial |
| RPL7 | 314.2710 | 0.4946 | 0.1460 | −3.3880 | 0.0007 | 0.0265 | NA |
| NDUFA3 | 150.9675 | 0.2713 | 0.0801 | −3.3880 | 0.0007 | 0.0265 | NA |
| MTR | 138.5731 | −0.1717 | 0.0507 | 3.3862 | 0.0007 | 0.0265 | Stromal |
| WNT11 | 8.9677 | −0.8533 | 0.2520 | 3.3857 | 0.0007 | 0.0265 | NA |
| TTC5 | 31.3741 | −0.2266 | 0.0669 | 3.3866 | 0.0007 | 0.0265 | NA |
| RPL28 | 1375.5966 | 0.2237 | 0.0661 | −3.3861 | 0.0007 | 0.0265 | Stromal |
| DEAF1 | 51.8180 | −0.2287 | 0.0676 | 3.3822 | 0.0007 | 0.0268 | Epithelial |
| TMCO1 | 354.7888 | 0.2536 | 0.0751 | −3.3787 | 0.0007 | 0.0271 | Epithelial |
| UNC13A | 4.3236 | −1.0060 | 0.2982 | 3.3740 | 0.0007 | 0.0275 | NA |
| PKM | 329.3862 | 0.2443 | 0.0724 | −3.3726 | 0.0007 | 0.0276 | Epithelial |
| TGFBR3 | 139.3440 | −0.4476 | 0.1328 | 3.3705 | 0.0008 | 0.0276 | Stromal |
| CYS1 | 25.3732 | −0.5641 | 0.1674 | 3.3710 | 0.0007 | 0.0276 | Stromal |
| AL109628.1 | 13.7715 | 0.3942 | 0.1169 | −3.3714 | 0.0007 | 0.0276 | NA |
| PPIA | 107.7663 | 0.2176 | 0.0646 | −3.3682 | 0.0008 | 0.0278 | Epithelial |
| CHMP3 | 225.4888 | 0.1441 | 0.0429 | −3.3623 | 0.0008 | 0.0282 | NA |
| ADGRB3 | 2.5631 | −1.0137 | 0.3014 | 3.3633 | 0.0008 | 0.0282 | Stromal |
| ASPH | 456.5756 | 0.3880 | 0.1154 | −3.3623 | 0.0008 | 0.0282 | Epithelial |
| AC002558.3 | 32.1220 | −0.3564 | 0.1060 | 3.3612 | 0.0008 | 0.0283 | Stromal |
| ABHD1 | 4.7078 | −0.4945 | 0.1472 | 3.3601 | 0.0008 | 0.0284 | NA |
| ARHGAP6 | 23.7311 | −0.3846 | 0.1145 | 3.3593 | 0.0008 | 0.0284 | Stromal |
| AL157838.1 | 1.9917 | −0.8755 | 0.2609 | 3.3562 | 0.0008 | 0.0287 | NA |
| PRC1 | 9.8601 | 0.5247 | 0.1564 | −3.3548 | 0.0008 | 0.0287 | Epithelial |
| GPM6A | 4.1254 | −1.0004 | 0.2983 | 3.3533 | 0.0008 | 0.0288 | NA |
| LMBR1L | 70.7537 | −0.2175 | 0.0649 | 3.3535 | 0.0008 | 0.0288 | Stromal |
| FIRRE | 16.1603 | 0.7192 | 0.2145 | −3.3526 | 0.0008 | 0.0288 | NA |
| HINT1 | 221.4778 | 0.2537 | 0.0757 | −3.3494 | 0.0008 | 0.0291 | Epithelial |
| ADIPOQ | 27.9307 | −0.8060 | 0.2410 | 3.3440 | 0.0008 | 0.0296 | Stromal |
| HIF3A | 4.8096 | −0.7224 | 0.2160 | 3.3441 | 0.0008 | 0.0296 | Stromal |
| EVC | 12.7339 | −0.4408 | 0.1319 | 3.3431 | 0.0008 | 0.0296 | Stromal |
| KAT2A | 82.5945 | −0.2847 | 0.0852 | 3.3424 | 0.0008 | 0.0296 | NA |
| CSMD1 | 2.6103 | −1.2330 | 0.3694 | 3.3380 | 0.0008 | 0.0300 | NA |
| MUCL1 | 1794.3958 | 1.2490 | 0.3742 | −3.3379 | 0.0008 | 0.0300 | Epithelial |
| GPR137C | 9.0497 | −0.5797 | 0.1738 | 3.3360 | 0.0008 | 0.0302 | NA |
| CD37 | 71.5520 | 0.5880 | 0.1764 | −3.3338 | 0.0009 | 0.0303 | Stromal |
| DOLPP1 | 13.0467 | 0.3560 | 0.1069 | −3.3299 | 0.0009 | 0.0307 | NA |
| ANKLE2 | 161.7664 | −0.1923 | 0.0577 | 3.3299 | 0.0009 | 0.0307 | NA |
| AC018362.1 | 4.6743 | −0.4961 | 0.1490 | 3.3293 | 0.0009 | 0.0307 | NA |
| LINC01348 | 2.9414 | 0.9803 | 0.2947 | −3.3264 | 0.0009 | 0.0307 | NA |
| FAM228B | 25.1765 | −0.2922 | 0.0878 | 3.3276 | 0.0009 | 0.0307 | NA |
| NOP10 | 54.2809 | 0.3166 | 0.0952 | −3.3267 | 0.0009 | 0.0307 | NA |
| NCCRP1 | 14.8823 | 1.1291 | 0.3393 | −3.3280 | 0.0009 | 0.0307 | Epithelial |
| TSPO | 197.8738 | 0.2935 | 0.0882 | −3.3265 | 0.0009 | 0.0307 | Epithelial |
| SDC1 | 293.6702 | 0.5674 | 0.1707 | −3.3250 | 0.0009 | 0.0308 | Epithelial |
| HLA-V | 1.4827 | 1.2364 | 0.3721 | −3.3225 | 0.0009 | 0.0310 | NA |
| HOXB3 | 65.2271 | −0.6660 | 0.2005 | 3.3223 | 0.0009 | 0.0310 | NA |
| MYDGF | 118.0437 | 0.2883 | 0.0868 | −3.3220 | 0.0009 | 0.0310 | NA |
| RN7SL4P | 6.2685 | −0.5236 | 0.1576 | 3.3213 | 0.0009 | 0.0310 | NA |
| CFAP69 | 38.9754 | −0.4118 | 0.1240 | 3.3202 | 0.0009 | 0.0310 | NA |
| CAPN15 | 12.8688 | 0.4493 | 0.1353 | −3.3204 | 0.0009 | 0.0310 | NA |
| AHNAK | 931.1495 | −0.2234 | 0.0673 | 3.3191 | 0.0009 | 0.0310 | Stromal |
| RBX1 | 102.8468 | 0.2296 | 0.0692 | −3.3195 | 0.0009 | 0.0310 | NA |
| BMP2K | 126.9361 | −0.3588 | 0.1082 | 3.3173 | 0.0009 | 0.0312 | NA |
| SOCS7 | 81.7016 | 0.5269 | 0.1590 | −3.3145 | 0.0009 | 0.0314 | Epithelial |
| HES6 | 9.5789 | 0.6781 | 0.2047 | −3.3130 | 0.0009 | 0.0316 | Epithelial |
| TAC1 | 11.8456 | −0.9722 | 0.2939 | 3.3081 | 0.0009 | 0.0321 | Stromal |
| GSTO1 | 97.6788 | 0.2543 | 0.0769 | −3.3064 | 0.0009 | 0.0321 | Stromal |
| RTCB | 96.1354 | 0.2008 | 0.0607 | −3.3065 | 0.0009 | 0.0321 | Epithelial |
| PMF1 | 57.4479 | 0.2684 | 0.0812 | −3.3051 | 0.0009 | 0.0322 | Epithelial |
| DNAJB11 | 71.0043 | 0.2093 | 0.0633 | −3.3043 | 0.0010 | 0.0323 | NA |
| TNS2 | 103.3789 | −0.2463 | 0.0746 | 3.3040 | 0.0010 | 0.0323 | Stromal |
| EIF3M | 82.0818 | 0.1742 | 0.0527 | −3.3027 | 0.0010 | 0.0323 | NA |
| LIG3 | 84.2878 | −0.2949 | 0.0893 | 3.3028 | 0.0010 | 0.0323 | Epithelial |
| ATP5MC3 | 215.1757 | 0.2181 | 0.0661 | −3.3018 | 0.0010 | 0.0323 | Epithelial |
| PRDM6 | 12.0138 | −0.6173 | 0.1870 | 3.3008 | 0.0010 | 0.0323 | Stromal |
| IFI27 | 586.8236 | −0.6902 | 0.2091 | 3.3009 | 0.0010 | 0.0323 | NA |
| COLEC12 | 98.3482 | −0.4106 | 0.1244 | 3.3003 | 0.0010 | 0.0323 | Stromal |
| HLA-DRB1 | 567.0707 | 0.4592 | 0.1392 | −3.2985 | 0.0010 | 0.0325 | Stromal |
| C18orf21 | 17.2745 | 0.2827 | 0.0857 | −3.2982 | 0.0010 | 0.0325 | NA |
| RBMS3 | 160.5167 | −0.2926 | 0.0888 | 3.2962 | 0.0010 | 0.0327 | Stromal |
| ATP5MGL | 5.0676 | 0.4768 | 0.1447 | −3.2950 | 0.0010 | 0.0327 | Stromal |
| SF3B4 | 8.1001 | 0.5431 | 0.1650 | −3.2914 | 0.0010 | 0.0329 | NA |
| DYNC1I2 | 184.0261 | 0.2111 | 0.0642 | −3.2914 | 0.0010 | 0.0329 | NA |
| AC005550.2 | 4.2622 | −0.6457 | 0.1961 | 3.2932 | 0.0010 | 0.0329 | NA |
| GGCT | 131.9023 | 0.3776 | 0.1147 | −3.2920 | 0.0010 | 0.0329 | Epithelial |
| TSIX | 7.2899 | −0.4714 | 0.1432 | 3.2922 | 0.0010 | 0.0329 | NA |
| KRT16 | 7.0167 | 0.7919 | 0.2407 | −3.2897 | 0.0010 | 0.0330 | Epithelial |
| CHD6 | 203.2044 | −0.2284 | 0.0694 | 3.2900 | 0.0010 | 0.0330 | Epithelial |
| NR2F2 | 463.3689 | −0.2579 | 0.0784 | 3.2891 | 0.0010 | 0.0330 | Stromal |
| TET1 | 28.3828 | −0.3892 | 0.1183 | 3.2886 | 0.0010 | 0.0330 | NA |
| RIC8B | 38.7839 | −0.3097 | 0.0942 | 3.2875 | 0.0010 | 0.0331 | NA |
| NPAS3 | 10.1017 | −0.5627 | 0.1712 | 3.2862 | 0.0010 | 0.0331 | NA |
| CLTC | 559.7193 | 0.3426 | 0.1042 | −3.2862 | 0.0010 | 0.0331 | Epithelial |
| HSPA12B | 19.8831 | −0.4223 | 0.1285 | 3.2869 | 0.0010 | 0.0331 | Stromal |
| RPL27A | 1819.9020 | 0.2186 | 0.0666 | −3.2820 | 0.0010 | 0.0335 | Stromal |
| UTP11 | 59.9357 | 0.1808 | 0.0551 | −3.2811 | 0.0010 | 0.0335 | NA |
| PELO | 21.8338 | 0.2990 | 0.0912 | −3.2787 | 0.0010 | 0.0338 | Stromal |
| AL049838.1 | 3.8978 | −0.7092 | 0.2165 | 3.2760 | 0.0011 | 0.0340 | Stromal |
| RECK | 25.3559 | −0.3615 | 0.1104 | 3.2747 | 0.0011 | 0.0341 | Stromal |
| TTC17 | 185.9962 | −0.1623 | 0.0495 | 3.2749 | 0.0011 | 0.0341 | NA |
| CALM2 | 473.1091 | 0.1973 | 0.0603 | −3.2714 | 0.0011 | 0.0343 | Epithelial |
| AC092620.1 | 18.6876 | −0.4847 | 0.1482 | 3.2713 | 0.0011 | 0.0343 | Epithelial |
| LMCD1-AS1 | 3.1337 | −0.7214 | 0.2206 | 3.2704 | 0.0011 | 0.0343 | NA |
| ITGA9-AS1 | 9.9439 | −0.4504 | 0.1377 | 3.2716 | 0.0011 | 0.0343 | NA |
| IMPDH2 | 187.4097 | 0.2461 | 0.0752 | −3.2708 | 0.0011 | 0.0343 | Epithelial |
| YY1AP1 | 116.6549 | −0.2519 | 0.0770 | 3.2696 | 0.0011 | 0.0344 | Epithelial |
| NOP58 | 210.4541 | 0.1743 | 0.0533 | −3.2668 | 0.0011 | 0.0346 | Epithelial |
| ATIC | 89.9696 | 0.2073 | 0.0635 | −3.2666 | 0.0011 | 0.0346 | Epithelial |
| KLK12 | 1.3456 | −2.2679 | 0.6951 | 3.2630 | 0.0011 | 0.0350 | Epithelial |
| ADAMTS5 | 102.5498 | −0.3929 | 0.1204 | 3.2631 | 0.0011 | 0.0350 | Stromal |
| NPIPB2 | 7.1652 | −0.5369 | 0.1646 | 3.2618 | 0.0011 | 0.0351 | NA |
| KRAS | 108.7721 | 0.2231 | 0.0684 | −3.2602 | 0.0011 | 0.0352 | NA |
| AL512770.1 | 5.3722 | −0.4772 | 0.1464 | 3.2592 | 0.0011 | 0.0353 | NA |
| ATP1B1 | 439.1032 | 0.4404 | 0.1352 | −3.2571 | 0.0011 | 0.0355 | Epithelial |
| UCP2 | 160.2975 | 0.3625 | 0.1114 | −3.2552 | 0.0011 | 0.0356 | NA |
| CBFA2T2 | 86.6974 | −0.2128 | 0.0654 | 3.2549 | 0.0011 | 0.0356 | Epithelial |
| RCOR3 | 133.4266 | −0.2582 | 0.0794 | 3.2531 | 0.0011 | 0.0358 | Epithelial |
| PCGF3 | 65.8560 | −0.2127 | 0.0654 | 3.2524 | 0.0011 | 0.0358 | NA |
| PSMA5 | 94.9886 | 0.2005 | 0.0617 | −3.2506 | 0.0012 | 0.0360 | Epithelial |
| RBM5 | 248.0450 | −0.2007 | 0.0617 | 3.2500 | 0.0012 | 0.0360 | NA |
| KMT5B | 164.0481 | −0.1920 | 0.0591 | 3.2483 | 0.0012 | 0.0362 | Epithelial |
| RGMB-AS1 | 3.1198 | 0.6735 | 0.2075 | −3.2461 | 0.0012 | 0.0364 | NA |
| AC073869.1 | 14.0922 | −0.4394 | 0.1354 | 3.2452 | 0.0012 | 0.0364 | NA |
| ATP5F1A | 317.2114 | 0.2051 | 0.0632 | −3.2452 | 0.0012 | 0.0364 | Epithelial |
| EID1 | 521.0178 | −0.1572 | 0.0485 | 3.2447 | 0.0012 | 0.0364 | Stromal |
| PLAGL1 | 66.6017 | −0.4008 | 0.1236 | 3.2429 | 0.0012 | 0.0365 | Stromal |
| BSPRY | 66.1595 | 0.4097 | 0.1263 | −3.2430 | 0.0012 | 0.0365 | Epithelial |
| ADHFE1 | 12.3683 | −0.4249 | 0.1310 | 3.2422 | 0.0012 | 0.0366 | Epithelial |
| RNU4-2 | 81.0032 | −0.2605 | 0.0804 | 3.2410 | 0.0012 | 0.0367 | NA |
| SNORD1B | 7.3125 | −0.5634 | 0.1738 | 3.2406 | 0.0012 | 0.0367 | NA |
| ATP13A4 | 21.8956 | 0.9382 | 0.2897 | −3.2384 | 0.0012 | 0.0369 | Epithelial |
| TP53BP1 | 160.5233 | −0.1937 | 0.0598 | 3.2379 | 0.0012 | 0.0369 | NA |
| OBSCN | 35.1556 | −0.3454 | 0.1067 | 3.2366 | 0.0012 | 0.0370 | NA |
| SMIM4 | 62.9278 | 0.2970 | 0.0918 | −3.2360 | 0.0012 | 0.0370 | Epithelial |
| PLIN1 | 60.4529 | −0.6839 | 0.2115 | 3.2341 | 0.0012 | 0.0372 | Stromal |
| SMC1A | 192.3521 | 0.1864 | 0.0576 | −3.2335 | 0.0012 | 0.0372 | Epithelial |
| VEGFD | 7.7826 | −0.8311 | 0.2571 | 3.2328 | 0.0012 | 0.0373 | Stromal |
| NPY1R | 183.3997 | −1.1260 | 0.3484 | 3.2323 | 0.0012 | 0.0373 | Epithelial |
| C1orf43 | 143.8168 | 0.2330 | 0.0721 | −3.2309 | 0.0012 | 0.0374 | Epithelial |
| SNHG16 | 112.9516 | 0.3272 | 0.1013 | −3.2298 | 0.0012 | 0.0375 | Epithelial |
| SRSF10 | 158.2835 | −0.1531 | 0.0474 | 3.2290 | 0.0012 | 0.0375 | NA |
| RPL22L1 | 49.1866 | 0.2790 | 0.0865 | −3.2253 | 0.0013 | 0.0379 | NA |
| ZNF136 | 37.3025 | −0.2138 | 0.0663 | 3.2253 | 0.0013 | 0.0379 | NA |
| AL450998.3 | 1.6989 | −0.7960 | 0.2470 | 3.2233 | 0.0013 | 0.0381 | NA |
| D2HGDH | 79.1685 | −0.2750 | 0.0853 | 3.2227 | 0.0013 | 0.0381 | Epithelial |
| RAP1B | 129.4365 | −0.2020 | 0.0627 | 3.2223 | 0.0013 | 0.0381 | Stromal |
| MTRNR2L5 | 10.3197 | −0.4389 | 0.1362 | 3.2217 | 0.0013 | 0.0381 | Epithelial |
| WDFY3-AS2 | 12.5303 | −0.3764 | 0.1169 | 3.2193 | 0.0013 | 0.0384 | Stromal |
| SLIRP | 131.3500 | 0.2082 | 0.0647 | −3.2191 | 0.0013 | 0.0384 | Epithelial |
| SLC9A7 | 64.8600 | 0.3072 | 0.0955 | −3.2160 | 0.0013 | 0.0387 | Epithelial |
| BCHE | 2.3468 | −0.9673 | 0.3010 | 3.2142 | 0.0013 | 0.0389 | NA |
| NDUFA8 | 55.3085 | 0.2421 | 0.0753 | −3.2139 | 0.0013 | 0.0389 | Epithelial |
| LDB2 | 47.0764 | −0.3352 | 0.1044 | 3.2114 | 0.0013 | 0.0391 | Stromal |
| TUFM | 249.4489 | 0.1842 | 0.0574 | −3.2113 | 0.0013 | 0.0391 | Epithelial |
| UBE2D2 | 144.0976 | 0.1382 | 0.0431 | −3.2095 | 0.0013 | 0.0393 | NA |
| UBOX5 | 28.2587 | −0.2360 | 0.0735 | 3.2095 | 0.0013 | 0.0393 | Epithelial |
| CD160 | 3.1491 | −0.5881 | 0.1834 | 3.2066 | 0.0013 | 0.0395 | NA |
| RPS15 | 127.4942 | 0.4835 | 0.1508 | −3.2063 | 0.0013 | 0.0395 | Epithelial |
| NOP53 | 486.6634 | 0.2256 | 0.0703 | −3.2069 | 0.0013 | 0.0395 | Stromal |
| HMGB1P5 | 8.3697 | 0.9913 | 0.3092 | −3.2058 | 0.0013 | 0.0395 | NA |
| PIK3C2A | 226.0220 | −0.1734 | 0.0541 | 3.2040 | 0.0014 | 0.0397 | NA |
| CFAP300 | 3.8412 | −0.7328 | 0.2288 | 3.2030 | 0.0014 | 0.0398 | NA |
| EIF2S2 | 114.6314 | 0.2177 | 0.0680 | −3.2026 | 0.0014 | 0.0398 | Epithelial |
| DENND4C | 76.1426 | −0.1832 | 0.0572 | 3.2019 | 0.0014 | 0.0398 | NA |
| HOXA11 | 1.6328 | 1.4662 | 0.4584 | −3.1984 | 0.0014 | 0.0402 | NA |
| ANKIB1 | 171.3588 | −0.1614 | 0.0505 | 3.1984 | 0.0014 | 0.0402 | NA |
| MRPL48 | 22.7613 | 0.3204 | 0.1002 | −3.1973 | 0.0014 | 0.0403 | NA |
| AL035409.1 | 2.9864 | −1.6108 | 0.5040 | 3.1960 | 0.0014 | 0.0404 | Epithelial |
| B4GALT3 | 72.3199 | 0.3100 | 0.0970 | −3.1945 | 0.0014 | 0.0406 | Epithelial |
| ULK1 | 86.3955 | −0.2127 | 0.0666 | 3.1938 | 0.0014 | 0.0406 | Epithelial |
| STS | 44.1307 | 0.4639 | 0.1453 | −3.1931 | 0.0014 | 0.0406 | NA |
| CLDN18 | 1.5511 | −0.7831 | 0.2453 | 3.1923 | 0.0014 | 0.0407 | NA |
| NOMO1 | 19.4955 | 0.3942 | 0.1235 | −3.1911 | 0.0014 | 0.0408 | NA |
| RN7SL792P | 11.2814 | −0.4715 | 0.1478 | 3.1909 | 0.0014 | 0.0408 | NA |
| KIAA2026 | 226.8925 | −0.1480 | 0.0464 | 3.1895 | 0.0014 | 0.0409 | Stromal |
| ZDHHC12 | 27.8956 | 0.2840 | 0.0891 | −3.1882 | 0.0014 | 0.0410 | Epithelial |
| RPS4X | 2023.6509 | 0.2320 | 0.0728 | −3.1879 | 0.0014 | 0.0410 | NA |
| RPS14 | 616.3547 | 0.3213 | 0.1009 | −3.1844 | 0.0015 | 0.0415 | NA |
| CCNB1IP1 | 30.5480 | 0.2908 | 0.0914 | −3.1804 | 0.0015 | 0.0420 | NA |
| RPS28 | 232.8941 | 0.4454 | 0.1401 | −3.1801 | 0.0015 | 0.0420 | Stromal |
| FBXW8 | 30.4072 | −0.2541 | 0.0800 | 3.1785 | 0.0015 | 0.0421 | Epithelial |
| UBTF | 170.2226 | −0.1409 | 0.0443 | 3.1783 | 0.0015 | 0.0421 | Stromal |
| EMC3 | 102.7446 | 0.1914 | 0.0603 | −3.1762 | 0.0015 | 0.0423 | Epithelial |
| NF1 | 178.2077 | −0.2249 | 0.0708 | 3.1764 | 0.0015 | 0.0423 | NA |
| KLHL11 | 4.8918 | −0.5626 | 0.1772 | 3.1756 | 0.0015 | 0.0423 | NA |
| CALY | 2.3295 | 1.3735 | 0.4326 | −3.1748 | 0.0015 | 0.0424 | NA |
| DLGAP2 | 2.4377 | −0.8196 | 0.2584 | 3.1722 | 0.0015 | 0.0425 | NA |
| RNA5SP378 | 1.1003 | −1.2588 | 0.3967 | 3.1731 | 0.0015 | 0.0425 | NA |
| SUZ12P1 | 41.6942 | −0.3270 | 0.1031 | 3.1719 | 0.0015 | 0.0425 | NA |
| RNU1-98P | 6.2849 | −0.5464 | 0.1722 | 3.1726 | 0.0015 | 0.0425 | NA |
| MT-CO3 | 10158.3779 | 0.2931 | 0.0924 | −3.1732 | 0.0015 | 0.0425 | Epithelial |
| MT-ND5 | 1421.6634 | 0.2803 | 0.0884 | −3.1717 | 0.0015 | 0.0425 | Epithelial |
| CNDP2 | 160.0044 | 0.2343 | 0.0739 | −3.1712 | 0.0015 | 0.0425 | Epithelial |
| REV1 | 97.4209 | −0.1579 | 0.0498 | 3.1702 | 0.0015 | 0.0426 | NA |
| SOX5 | 16.2232 | −0.4595 | 0.1450 | 3.1697 | 0.0015 | 0.0426 | Stromal |
| AC068580.4 | 2.2304 | −0.8723 | 0.2752 | 3.1691 | 0.0015 | 0.0426 | NA |
| DNAJC3 | 180.2157 | 0.2301 | 0.0726 | −3.1689 | 0.0015 | 0.0426 | NA |
| B3GALT5-AS1 | 5.0078 | −0.9341 | 0.2949 | 3.1680 | 0.0015 | 0.0427 | NA |
| XRCC5 | 383.5691 | 0.1333 | 0.0421 | −3.1652 | 0.0015 | 0.0430 | Epithelial |
| C16orf54 | 9.4596 | 0.6357 | 0.2009 | −3.1643 | 0.0016 | 0.0430 | Stromal |
| RPS6KB1 | 95.6182 | 0.3690 | 0.1166 | −3.1645 | 0.0016 | 0.0430 | Epithelial |
| ZDHHC17 | 71.6643 | −0.2340 | 0.0740 | 3.1636 | 0.0016 | 0.0430 | Stromal |
| CSAD | 222.4910 | −0.3054 | 0.0966 | 3.1622 | 0.0016 | 0.0432 | Epithelial |
| AC011379.2 | 9.4042 | −0.5066 | 0.1603 | 3.1608 | 0.0016 | 0.0434 | NA |
| SEMA7A | 4.3260 | 0.6326 | 0.2002 | −3.1598 | 0.0016 | 0.0434 | Stromal |
| APBB2 | 118.3163 | −0.2985 | 0.0945 | 3.1580 | 0.0016 | 0.0436 | NA |
| WDR5 | 30.8305 | 0.2453 | 0.0778 | −3.1535 | 0.0016 | 0.0443 | NA |
| CHCHD4 | 12.6747 | 0.3391 | 0.1076 | −3.1525 | 0.0016 | 0.0443 | NA |
| MYH9 | 809.5347 | 0.1636 | 0.0519 | −3.1527 | 0.0016 | 0.0443 | Stromal |
| MKRN2 | 27.9893 | 0.2228 | 0.0707 | −3.1504 | 0.0016 | 0.0445 | NA |
| AL022342.1 | 1.7947 | 0.7074 | 0.2247 | −3.1488 | 0.0016 | 0.0447 | NA |
| AP003086.2 | 1.7086 | −0.8103 | 0.2576 | 3.1456 | 0.0017 | 0.0450 | NA |
| PHLDB1 | 145.5016 | −0.2265 | 0.0720 | 3.1463 | 0.0017 | 0.0450 | Stromal |
| LYRM9 | 28.5390 | −0.2987 | 0.0950 | 3.1456 | 0.0017 | 0.0450 | Stromal |
| CSKMT | 54.1899 | 0.4264 | 0.1356 | −3.1448 | 0.0017 | 0.0451 | Epithelial |
| RILPL1 | 38.3165 | −0.2167 | 0.0689 | 3.1444 | 0.0017 | 0.0451 | Stromal |
| TAL1 | 7.7780 | −0.3677 | 0.1171 | 3.1411 | 0.0017 | 0.0452 | Stromal |
| ANGEL2 | 51.1979 | −0.1912 | 0.0609 | 3.1405 | 0.0017 | 0.0452 | NA |
| RN7SKP55 | 15.9280 | −0.4900 | 0.1560 | 3.1413 | 0.0017 | 0.0452 | NA |
| EIPR1 | 17.4270 | 0.2596 | 0.0826 | −3.1413 | 0.0017 | 0.0452 | Epithelial |
| NBEAL1 | 126.9912 | −0.1620 | 0.0515 | 3.1423 | 0.0017 | 0.0452 | NA |
| MANF | 118.7692 | 0.2485 | 0.0791 | −3.1405 | 0.0017 | 0.0452 | NA |
| AC087239.1 | 2.2180 | 0.6081 | 0.1936 | −3.1408 | 0.0017 | 0.0452 | Epithelial |
| AL049780.1 | 2.2465 | −0.8073 | 0.2570 | 3.1416 | 0.0017 | 0.0452 | NA |
| RSL1D1 | 325.9444 | 0.1669 | 0.0532 | −3.1401 | 0.0017 | 0.0452 | Epithelial |
| ACTR1B | 51.7867 | 0.1888 | 0.0601 | −3.1395 | 0.0017 | 0.0452 | Epithelial |
| RPLP2 | 1424.5660 | 0.3192 | 0.1017 | −3.1391 | 0.0017 | 0.0452 | Stromal |
| TWF2 | 47.2722 | 0.2417 | 0.0771 | −3.1367 | 0.0017 | 0.0455 | Stromal |
| PCSK1 | 5.5923 | −1.3016 | 0.4150 | 3.1363 | 0.0017 | 0.0456 | NA |
| AC011815.2 | 3.2884 | −0.7209 | 0.2299 | 3.1353 | 0.0017 | 0.0456 | NA |
| RPL5 | 832.3017 | 0.2382 | 0.0760 | −3.1327 | 0.0017 | 0.0459 | Stromal |
| CCT4 | 109.1307 | 0.1827 | 0.0583 | −3.1328 | 0.0017 | 0.0459 | Epithelial |
| MT-TV | 169.4806 | 0.5299 | 0.1691 | −3.1334 | 0.0017 | 0.0459 | Epithelial |
| ARF1 | 327.8007 | 0.2142 | 0.0684 | −3.1319 | 0.0017 | 0.0459 | Epithelial |
| PIKFYVE | 73.5372 | −0.1661 | 0.0531 | 3.1298 | 0.0017 | 0.0462 | Stromal |
| CTBP1-DT | 29.7469 | −0.2551 | 0.0815 | 3.1291 | 0.0018 | 0.0462 | Epithelial |
| CLDN4 | 269.5882 | 0.3588 | 0.1147 | −3.1283 | 0.0018 | 0.0462 | Epithelial |
| HECTD4 | 150.1558 | −0.2069 | 0.0661 | 3.1283 | 0.0018 | 0.0462 | Epithelial |
| FBL | 216.3621 | 0.2055 | 0.0657 | −3.1282 | 0.0018 | 0.0462 | NA |
| PTOV1 | 109.4092 | 0.2562 | 0.0819 | −3.1272 | 0.0018 | 0.0463 | Epithelial |
| CTNNBIP1 | 60.7606 | 0.3139 | 0.1006 | −3.1206 | 0.0018 | 0.0469 | Epithelial |
| FBXO42 | 63.3500 | −0.1718 | 0.0550 | 3.1225 | 0.0018 | 0.0469 | NA |
| MAEL | 1.9251 | −0.9853 | 0.3157 | 3.1206 | 0.0018 | 0.0469 | NA |
| REEP5 | 300.7409 | 0.2332 | 0.0747 | −3.1207 | 0.0018 | 0.0469 | Epithelial |
| UBN2 | 178.2958 | −0.2065 | 0.0662 | 3.1215 | 0.0018 | 0.0469 | Epithelial |
| GPT2 | 30.9993 | 0.4760 | 0.1525 | −3.1207 | 0.0018 | 0.0469 | Epithelial |
| NAGS | 5.3729 | −0.8211 | 0.2630 | 3.1225 | 0.0018 | 0.0469 | NA |
| AL133520.1 | 1.3277 | 0.8235 | 0.2638 | −3.1215 | 0.0018 | 0.0469 | NA |
| CLHC1 | 30.3029 | −0.3171 | 0.1017 | 3.1183 | 0.0018 | 0.0470 | Epithelial |
| SEMA3D | 15.5348 | −0.4202 | 0.1348 | 3.1180 | 0.0018 | 0.0470 | Stromal |
| AC115837.1 | 1.1701 | −0.8140 | 0.2609 | 3.1194 | 0.0018 | 0.0470 | NA |
| PPFIA1 | 216.4723 | −0.3010 | 0.0966 | 3.1176 | 0.0018 | 0.0470 | Epithelial |
| ITCH | 136.2446 | −0.1919 | 0.0615 | 3.1191 | 0.0018 | 0.0470 | NA |
| AL022476.1 | 1.1179 | −0.9935 | 0.3187 | 3.1177 | 0.0018 | 0.0470 | NA |
| F11R | 134.6530 | 0.2807 | 0.0901 | −3.1160 | 0.0018 | 0.0470 | Epithelial |
| CIDEC | 15.4688 | −0.7467 | 0.2396 | 3.1162 | 0.0018 | 0.0470 | Stromal |
| AC010623.1 | 1.4559 | −0.9396 | 0.3015 | 3.1166 | 0.0018 | 0.0470 | NA |
| GPD1 | 33.9058 | −0.7142 | 0.2292 | 3.1159 | 0.0018 | 0.0470 | Stromal |
| COX5B | 163.4785 | 0.2012 | 0.0646 | −3.1151 | 0.0018 | 0.0470 | Epithelial |
| ICA1L | 25.1031 | −0.3428 | 0.1101 | 3.1150 | 0.0018 | 0.0470 | Stromal |
| BCR | 9.3406 | 0.4409 | 0.1416 | −3.1143 | 0.0018 | 0.0470 | NA |
| TSPAN9 | 55.7077 | 0.2617 | 0.0841 | −3.1125 | 0.0019 | 0.0473 | NA |
| SEC61A1 | 229.2580 | 0.2002 | 0.0644 | −3.1097 | 0.0019 | 0.0475 | NA |
| OMD | 21.1451 | −0.4149 | 0.1334 | 3.1099 | 0.0019 | 0.0475 | Stromal |
| TUBB4B | 149.4579 | 0.3123 | 0.1004 | −3.1101 | 0.0019 | 0.0475 | Epithelial |
| TPT1 | 1957.7779 | 0.2491 | 0.0801 | −3.1099 | 0.0019 | 0.0475 | Stromal |
| ATG2B | 47.6423 | −0.2091 | 0.0673 | 3.1082 | 0.0019 | 0.0477 | NA |
| MTND3P17 | 1.1783 | −0.7924 | 0.2551 | 3.1067 | 0.0019 | 0.0478 | NA |
| BID | 29.4510 | 0.2850 | 0.0917 | −3.1066 | 0.0019 | 0.0478 | Stromal |
| POLRMT | 25.0831 | 0.2492 | 0.0802 | −3.1061 | 0.0019 | 0.0478 | NA |
| TRPM6 | 4.3285 | −0.6579 | 0.2119 | 3.1049 | 0.0019 | 0.0478 | Stromal |
| STN1 | 29.7006 | 0.2412 | 0.0777 | −3.1056 | 0.0019 | 0.0478 | NA |
| SHMT2 | 65.3636 | 0.3366 | 0.1084 | −3.1051 | 0.0019 | 0.0478 | NA |
| STOX2 | 18.9472 | −0.4132 | 0.1331 | 3.1032 | 0.0019 | 0.0479 | Stromal |
| SOWAHA | 11.1448 | −0.7872 | 0.2537 | 3.1028 | 0.0019 | 0.0479 | Epithelial |
| MALAT1 | 48925.2279 | −0.4062 | 0.1309 | 3.1029 | 0.0019 | 0.0479 | Epithelial |
| ARPP19 | 119.1772 | 0.1786 | 0.0575 | −3.1038 | 0.0019 | 0.0479 | Epithelial |
| KANTR | 32.9643 | −0.2841 | 0.0915 | 3.1041 | 0.0019 | 0.0479 | NA |
| PDZK1IP1 | 56.4222 | 0.7729 | 0.2492 | −3.1020 | 0.0019 | 0.0479 | Epithelial |
| AC011503.1 | 2.0343 | −1.0850 | 0.3500 | 3.1005 | 0.0019 | 0.0481 | Stromal |
| SNHG19 | 84.6142 | 0.3782 | 0.1221 | −3.0982 | 0.0019 | 0.0484 | Epithelial |
| TBK1 | 53.9059 | −0.1917 | 0.0619 | 3.0975 | 0.0020 | 0.0485 | NA |
| PNPLA7 | 35.9658 | −0.3046 | 0.0984 | 3.0960 | 0.0020 | 0.0485 | NA |
| SRSF8 | 65.6018 | 0.2047 | 0.0661 | −3.0961 | 0.0020 | 0.0485 | NA |
| AP1B1 | 117.1570 | 0.2154 | 0.0696 | −3.0963 | 0.0020 | 0.0485 | NA |
| SUN2 | 88.1320 | 0.2355 | 0.0761 | −3.0943 | 0.0020 | 0.0488 | Stromal |
| DIP2C | 73.3586 | −0.1890 | 0.0611 | 3.0934 | 0.0020 | 0.0489 | Stromal |
| TBCA | 168.7333 | 0.2115 | 0.0684 | −3.0924 | 0.0020 | 0.0489 | Epithelial |
| FAM193B | 92.6232 | −0.2106 | 0.0681 | 3.0927 | 0.0020 | 0.0489 | NA |
| CFL1 | 1550.3793 | 0.1616 | 0.0523 | −3.0900 | 0.0020 | 0.0492 | Epithelial |
| LSM4 | 152.3750 | 0.2299 | 0.0744 | −3.0900 | 0.0020 | 0.0492 | Epithelial |
| SRM | 134.5017 | 0.2260 | 0.0732 | −3.0890 | 0.0020 | 0.0492 | NA |
| AC044787.1 | 2.7654 | 0.6419 | 0.2078 | −3.0894 | 0.0020 | 0.0492 | NA |
| FOXRED2 | 46.8172 | 0.3135 | 0.1015 | −3.0887 | 0.0020 | 0.0492 | NA |
| ARMCX1 | 34.1835 | −0.3304 | 0.1070 | 3.0879 | 0.0020 | 0.0493 | NA |
| PSMD14 | 94.8451 | 0.1765 | 0.0572 | −3.0833 | 0.0020 | 0.0498 | Epithelial |
| ZZEF1 | 76.8722 | −0.2104 | 0.0682 | 3.0836 | 0.0020 | 0.0498 | Stromal |
| AC079336.5 | 2.7864 | −0.6102 | 0.1979 | 3.0834 | 0.0020 | 0.0498 | Stromal |
| AP2M1 | 235.2423 | 0.1484 | 0.0481 | −3.0825 | 0.0021 | 0.0499 | NA |
| PPA1 | 71.3369 | 0.2360 | 0.0766 | −3.0818 | 0.0021 | 0.0500 | NA |

log2FoldChange > 0: Up in ipsilateral breast event (either DCIS or IBC) within 5 years. Compartment column indicates if the respective gene was significantly differentially expressed (FDR < 0.05) in the epithelial or stromal compartment by DESeq2 analysis of stromal vs epithelial RAHBT LCM samples.

### Tissue Processing Systems

Systems useful to carry out the methods of tissue processing as described herein can be implemented in hardware, software, firmware, or combinations of hardware, software and/or firmware. In some examples, the systems may be implemented using a non-transitory computer readable medium storing computer executable instructions that when executed by one or more processors of a computer cause the computer to perform operations. Computer readable media suitable for implementing the systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system (e.g., comprising genes and/or classifiers as taught herein) may be located on a single device or computing platform or may be distributed across multiple devices or computing platforms.

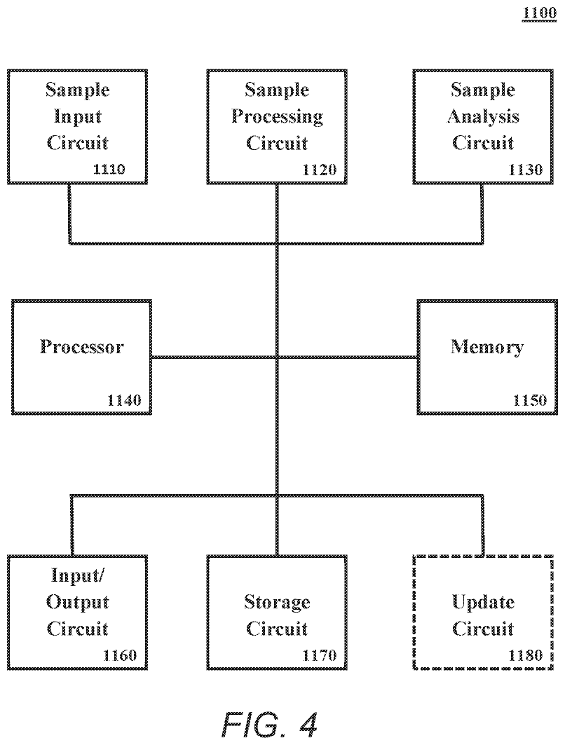

With reference to FIG. 4, a tissue processing system and/or computer program product 1100 may be used according to various embodiments described herein. A tissue processing system and/or computer program product 1100 may be embodied as one or more enterprise, application, personal, pervasive and/or embedded computer systems that are operable to receive, transmit, process and store data using any suitable combination of software, firmware and/or hardware and that may be standalone and/or interconnected by any conventional, public and/or private, real and/or virtual, wired and/or wireless network including all or a portion of the global communication network known as the Internet, and may include various types of tangible, non-transitory computer readable medium.

As shown in FIG. 4, the tissue processing system 1100 may include a processor subsystem 1140, including one or more Central Processing Units (CPU) on which one or more operating systems and/or one or more applications run. While one processor 1140 is shown, it will be understood that multiple processors 1140 may be present, which may be either electrically interconnected or separate. Processor(s) 1140 are configured to execute computer program code from memory devices, such as memory subsystem 1150, to perform at least some of the operations and methods described herein, and may be any conventional or special purpose processor, including, but not limited to, digital signal processor (DSP), field programmable gate array (FPGA), application specific integrated circuit (ASIC), and multi-core processors.

The memory subsystem 1150 may include a hierarchy of memory devices such as Random Access Memory (RAM), Read-Only Memory (ROM), Erasable Programmable Read-Only Memory (EPROM) or flash memory, and/or any other solid state memory devices. A storage circuit 1170 may also be provided, which may include, for example, a portable computer diskette, a hard disk, a portable Compact Disk Read-Only Memory (CDROM), an optical storage device, a magnetic storage device and/or any other kind of disk- or tape-based storage subsystem. The storage circuit 1170 may provide non-volatile storage of data/parameters/classifiers for the tissue processing system 1100. The storage circuit 1170 may include disk drive and/or network store components. The storage circuit 1170 may be used to store code to be executed and/or data to be accessed by the processor 1140. In some embodiments, the storage circuit 1170 may store databases which provide access to the data/parameters/classifiers used for the tissue processing system 1110 such as the list of genes, weights, thresholds, etc. Any combination of one or more computer readable media may be utilized by the storage circuit 1170. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. As used herein, a computer readable storage medium may be any tangible medium that can contain, or store a program for use by or in connection with an instruction execution system, apparatus, or device.

An input/output circuit 1160 may include displays and/or user input devices, such as keyboards, touch screens and/or pointing devices. Devices attached to the input/output circuit 1160 may be used to provide information to the processor 1140 by a user of the tissue processing system 1100. Devices attached to the input/output circuit 1160 may include networking or communication controllers, input devices (keyboard, a mouse, touch screen, etc.) and output devices (printer or display). The input/output circuit 1160 may also provide an interface to devices, such as a display and/or printer, to which results of the operations of the tissue processing system 1100 can be communicated so as to be provided to the user of the tissue processing system 1100.

An optional update circuit 1180 may be included as an interface for providing updates to the tissue processing system 1100. Updates may include updates to the code executed by the processor 1140 that are stored in the memory subsystem 1150 and/or the storage circuit 1170. Updates provided via the update circuit 1180 may also include updates to portions of the storage circuit 1170 related to a database and/or other data storage format which maintains information for the tissue processing system 1100, such as the signatures, weights, thresholds, etc.

The sample input circuit 1110 of the tissue processing system 1100 may provide an interface for the platform as described hereinabove to receive tissue samples to be analyzed. The sample input circuit 1110 may include mechanical elements, as well as electrical elements, which receive a tissue sample provided by a user to the tissue processing system 1100 and transport the tissue sample within the tissue processing system 1100 and/or platform to be processed. The sample input circuit 1110 may include a bar code reader that identifies a bar-coded container for identification of the sample and/or test order form. The sample processing circuit 1120 may further process the tissue sample within the tissue processing system 1100 and/or platform so as to prepare the sample for automated analysis. The sample analysis circuit 1130 may automatically analyze the processed tissue sample. The sample analysis circuit 1130 may be used in measuring, e.g., gene expression levels of a pre-defined set of genes with the tissue sample provided to the tissue processing system 1100. The sample analysis circuit 1130 may also optionally generate normalized gene expression values by normalizing the gene expression levels. The sample analysis circuit 1130 may retrieve from the storage circuit 1170 a DCIS classifier as taught herein. The sample analysis circuit 1130 may enter the gene expression values into the classifier. The sample analysis circuit 1130 may calculate a score or probability of DCIS recurrence and/or progression based upon said classifier, via the input/output circuit 1160.

The sample input circuit 1110, the sample processing circuit 1120, the sample analysis circuit 1130, the input/output circuit 1160, the storage circuit 1170, and/or the update circuit 1180 may execute at least partially under the control of the one or more processors 1140 of the tissue processing system 1100. As used herein, executing “under the control” of the processor 1140 means that the operations performed by the sample input circuit 1110, the sample processing circuit 1120, the sample analysis circuit 1130, the input/output circuit 1160, the storage circuit 1170, and/or the update circuit 1180 may be at least partially executed and/or directed by the processor 1140, but does not preclude at least a portion of the operations of those components being separately electrically or mechanically automated. The processor 1140 may control the operations of the tissue processing system 1100, as described herein, via the execution of computer program code.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PUP, ABAP, dynamic programming languages such as Python, Ruby and Groovy, or other programming languages. The program code may execute entirely on the tissue processing system 1100, partly on the tissue processing system 1100, as a stand-alone software package, partly on the tissue processing system 1100 and partly on a remote computer or entirely on the remote computer or server. In the latter scenario, the remote computer may be connected to the tissue processing system 1100 through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

The present invention is further described in the following non-limiting examples.

### EXAMPLES

Here, as part of the Human Tumor Atlas Network (HTAN) we present two DCIS cohorts, the Translational Breast Cancer Research Consortium (TBCRC) 038 study and the Resource of Archival Breast Tissue (RAHBT), for multimodal molecular analyses. We performed comprehensive integrated molecular profiling of these complementary, clinically annotated, longitudinally sampled cohorts, to understand the spectrum of molecular changes in DCIS and to identify both tumor and stromal predictors of subsequent events. We used multidimensional and multiparametric approaches to address central conceptual themes of cancer progression, ecology, and evolutionary biology. The breast precancer atlas (PCA) presented here may facilitate phylogenetic analysis to reconstruct the relationship between DCIS and IBC, the natural history of DCIS, and factors that underlie progression to invasive disease.

### Results

### Study Design and Cohorts

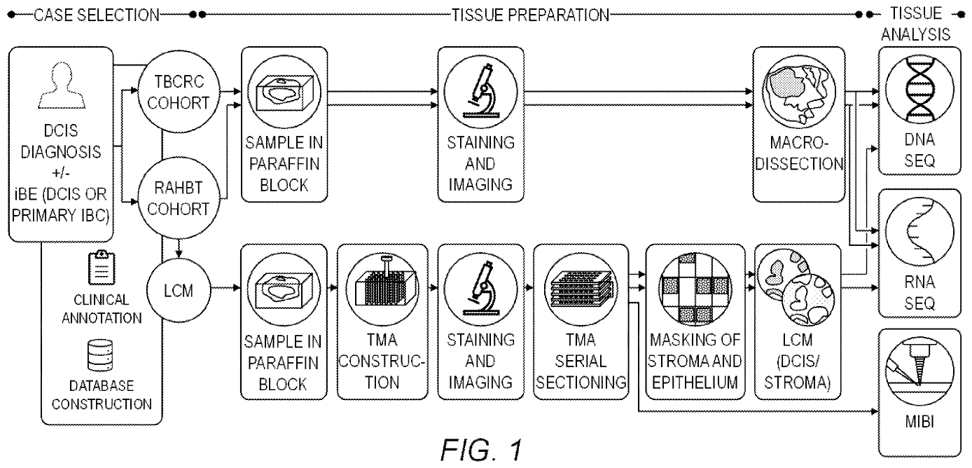

We generated two retrospective case-control cohorts of patients initially diagnosed with pure DCIS with or without a subsequent ipsilateral breast event (iBE, either DCIS or invasive breast cancer (IBC)) after surgical treatment. Identical eligibility criteria were used for outcome analysis in both cohorts. The RAHBT cohort used for outcome analysis has 97 cases with median diagnosis at age 53, and 40 months median time to recurrence. Over half (66.0%) had lumpectomy with radiation, 10.3% had lumpectomy without radiation, and 35% were identified as black. The TBCRC cohort included 216 patients with median diagnosis at age 52, and 48 months median time to recurrence. More than half (55.5%) had lumpectomy with radiation, 15.3% had lumpectomy without radiation, and 30.0% were identified as black. FIG. 1 shows an outline of cohorts and analyses in this study. Cohort descriptions are provided in Table 2.

TABLE 2
Breast Pre-cancer Atlas Patient Cohorts with RNA-seq data and
ipsilateral breast event (iBE) used for outcome analysis.

| | TBCRC | | | RAHBT | | |
|---|---|---|---|---|---|---|
| | DCIS | DCIS with | DCIS with | DCIS | DCIS with | DCIS with |
| | without | DCIS | Invasive | without | DCIS | Invasive |
| | recurrence | Recurrence | Recurrence | recurrence | Recurrence | Recurrence |
| | (N = 95) | (N = 66) | (N = 55) | (N = 68) | (N = 15) | (N = 14) |
| Year of | | | | | | |
| Diagnosis | | | | | | |
| Median | 2009 | 2008 | 2006 | 2006 | 2008 | 2009 |
| Age at | | | | | | |
| Diagnosis | | | | | | |
| Median | 54 | 54 | 50 | 52 | 53 | 52 |
| Mean (±SD) | 54.4 (±8.5) | 55.2 (±9.8) | 52.6 (±9.8) | 53.1 (±7.2) | 52., 5(±6.0) | 55.1(±11.1) |
| Grade | | | | | | |
| 1 | 5 [5.3%] | 6 [9.0%] | 3 [5.5%] | 18 [26.5%] | 4 [26.7%] | 3 [21.4%] |
| 2 | 37 38.9%] | 26 [39.4%] | 19 [34.5%] | 28 [48.2%] | 4 [26.7%] | 8 [57.1%] |
| 3 | 53 55.8%] | 34 [51.5%] | 33 [60.0%] | 22 [32.4%] | 7 [46.7%] | 2 [21.4%] |
| Pathologic | | | | | | |
| Tumor Size | | | | | | |
| Median | 2.1 | 1.5 | 1.9 | | | |
| Mean (±SD) | 2.7 (±1.9) | 2.2 (± 2.0) | 2.8 (± 2.6) | | | |
| Marker | | | | | | |
| Status | | | | | | |
| ER(+) | 60 [63.2%] | 41 [62.1%] | 37 [67.3%] | 55 [80.9%] | 8 [53.3%] | 12 [85.7%] |
| ER(−) | 35 [36.8%] | 25 [37.9%] | 18 [32.7%] | 13 [19.1%] | 7 [46.7%] | 2 [14.3%] |
| ER(+) Dx | 0 | 2 [3.0%] | 4 [7.3%] | 3 [4.4%] | 0 | 3 [21.4%] |
| before 2000 | | | | | | |
| ER(+) Dx | 60 [63.2%] | 39 [59.1%] | 33 [60.0%] | 52 [76.5%] | 8 [53.3%] | 9 [64.3%] |
| 2000 & after | | | | | | |
| ER(−) Dx | 0 | 0 | 1 [1.8%] | 2 [2.9%] | 2 [13.3%] | 0 |
| before 2000 | | | | | | |
| ER(−) Dx | 35 [36.8%] | 25 [37.9%] | 17 [30.9%] | 11 [16.2%] | 5 [33.3%] | 2 [14.3%] |
| 2000 & after | | | | | | |
| Treatment | | | | | | |
| Lumpectomy + | 58 [61.1%] | 40 [60.6%] | 22 [40.0%] | 6 [8.8%] | 2 [13.3%] | 2 [14.3%] |
| Radiation | | | | | | |
| Lumpectomy − | 5 [5.3%] | 16 [25.2%] | 12 [21.8%] | 45 [66.2%] | 11 [73.3%] | 8 [57.1%] |
| Radiation | | | | | | |
| Lumpectomy | 1 [1.1%] | 1 [1.5%] | 2 [3.6%] | 0 | 0 | 0 |
| Radiation | | | | | | |
| Unknown | | | | | | |
| Mastectomy | 31 [32.6%] | 9 [13.6%] | 19 [34.5%] | 17 [25.0%] | 2 [13.3%] | 4 [28.6%] |

Time to Recurrence* (months)

| | | | | | | |
|---|---|---|---|---|---|---|
| Mean (±SD) | 105.7 (± | 52.7 (±39.9) | 71.2 (±43.9) | 139.8 (±52.7) | 54.9 (± | 73.4 (±68.4) |
| | 37.0) | | | | 40.4) | |
| Median | 96 | 40 | 58 | 141 | 36 | 47 |
| Margins | | | | | | |
| Ink on | 0 | 0 | 0 | 0 | 0 | 0 |
| tumor | | | | | | |
| <2 mm | 27 | 28 | 17 | 15 | 4 [26.7%] | 6 [42.9%] |
| | [28.4%] | [42.4%] | [30.9%] | [22.1%] | | |
| At least≥ | 37 | 25 | 21 | 11 | 4 [26.7%] | 1 [7.1%] |
| 2 mm | [38.9%] | [37.9%] | [38.2%] | [16.2%] | | |
| Clear, | 31 | 13 | 17 | 42 | 7 [46.7%] | 7 [50.0%] |
| unknown | [32.6%] | [19.7%] | [30.9%] | [61.8%] | | |
| mm | | | | | | |
| Race | | | | | | |
| White | 62 | 38 | 28 | 44 | 10 | 9 [64.3%] |
| | [65.2%] | [57.6%] | [50.9%] | [64.7%] | [66.7%] | |
| Black | 22 | 21 | 22 | 24 | 5 [33.3%] | 5 [35.7%] |
| | [23.2%] | [31.8%] | [40.0%] | [35.3%] | | |
| Asian | 2 [2.1%] | 1 [1.5%] | 2 [3.6%] | 0 | 0 | 0 |
| Pacific | 0 | 1 [1.5%] | 0 | 0 | 0 | 0 |
| Islander | | | | | | |
| Other | 0 | 0 | 0 | 0 | 0 | 0 |
| Unknown | 9 [9.5%] | 5 [7.6%] | 3 [5.5%] | 0 | 0 | 0 |

*To end of follow-up for no recurrence.

### Prognostic Classifier Predicts Early Recurrence

The TBCRC and RAHBT cohorts were designed to investigate biological determinants of recurrence by matching patients with subsequent iBE to patients that did not have any events during long-term follow-up.

To identify gene expression patterns correlating with outcome, we analyzed RNA from primary DCIS with iBEs within 5 years vs the remaining samples in TBCRC, to avoid including non-clonal events that might be more common in later years. We identified 812 differentially expressed (DE) genes at 0.05 false discovery rate (FDR). Table 1 above lists 812 differentially expressed genes from DESeq2 analysis iBEs within 5 years vs. the rest in TBCRC.

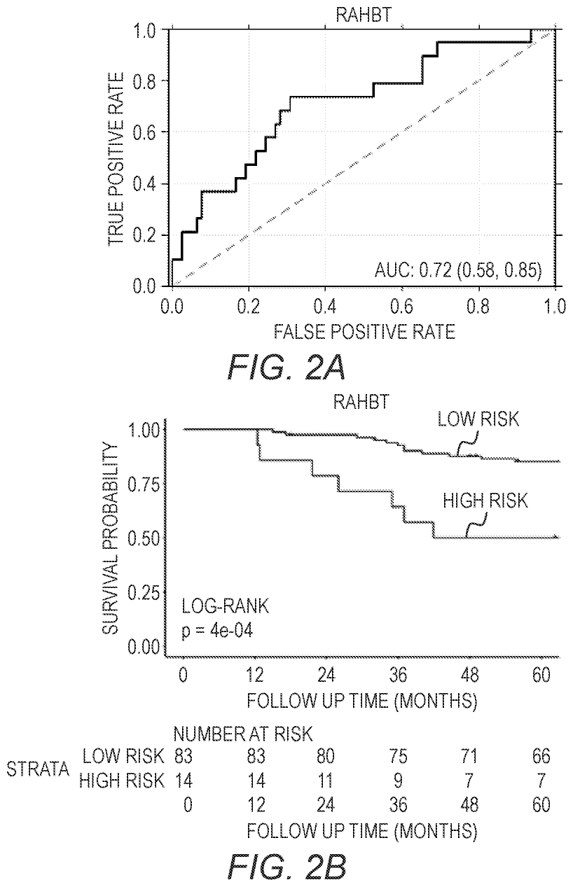

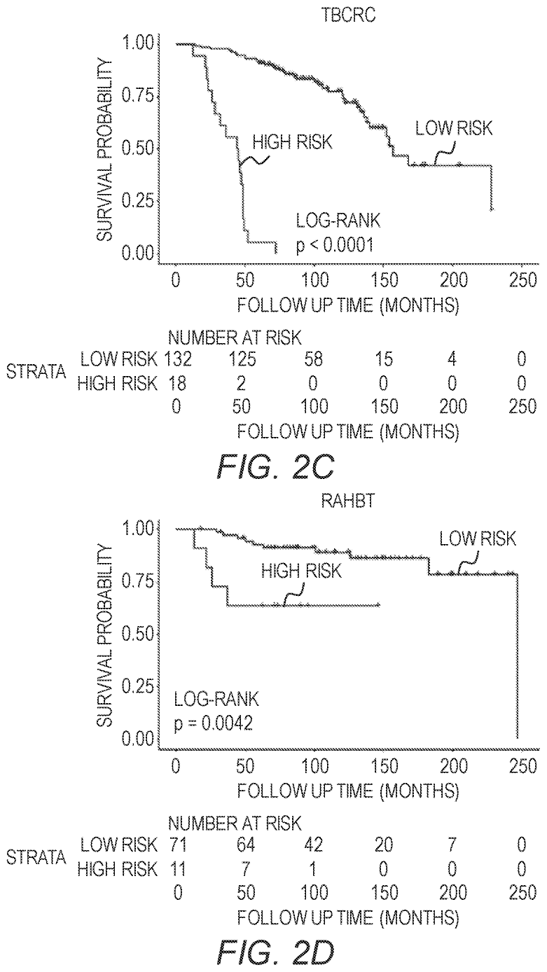

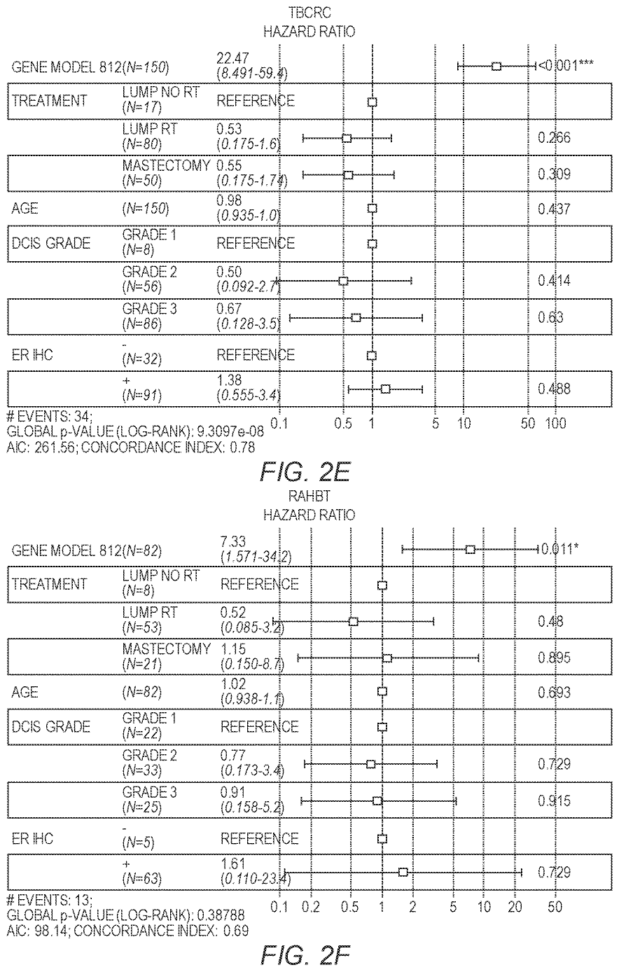

To identify copy number aberrations (CNAs) that correlate with outcome, we performed light-pass whole genome sequencing (WGS) on DNA from FFPE samples in both cohorts (n=228). We identified 29 recurrent CNAs across both cohorts, none of which were predictive of recurrence. Given the absence of significant CNAs, we trained a Random Forest classifier in TBCRC using only the 812 DE genes. The classifier was validated in RAHBT, with an ROC AUC of 0.72 (FIG. 2A), Precision 0.86, Recall 0.91, and F1 score 0.88, indicating that the classifier performed well also in the test cohort. The classifier significantly predicted any subsequent iBE in both cohorts (RAHBT P=0.0004, FIG. 2B). Importantly, it was also a significant predictor of invasive iBEs over the full follow-up time (TBCRC P<0.0001, RAHBT P=0.0042, FIGS. 2C-2D), demonstrating the classifier could specifically identify DCIS that progress to IBC.

Next, we examined whether the 812 gene classifier remained an independent predictor of outcome when combined with clinical features. We performed multivariable Cox regression analysis including the classifier, treatment, age, clinical ER, and DCIS grade. While multivariable analysis demonstrated a trend for treatment type and ER status for outcome, only the 812 gene classifier was significant in both cohorts (RAHBT HR=3.48, (95% CI: 1.14-10.6), P=0.028). Importantly, in multivariable analysis for invasive iBEs only, the classifier showed an even stronger prognostic value in both cohorts, with a hazard ratio of 7.33 in RAHBT (95% CI: 1.57-34.2, P=0.011, FIGS. 2E-2F). While previous studies found association between ER status and DCIS outcome, Kaplan-Meier analysis of clinical ER status (IHC-based) demonstrated a trend in RAHBT (P=0.053), but not in TBCRC (P=0.2). Moreover, the 812 gene classifier showed no prognostic value for progression free disease or overall survival for 1064 IBCs from The Cancer Genome Atlas (TCGA), suggesting that the classifier is specific for the DCIS stage.

To compare the 812 gene classifier to commercially available prognostic tests for DCIS, we calculated the Oncotype DCIS score as previously described using TBCRC and RAHBT RNA-sequencing data. We found that, in contrast to the 812 gene classifier, the DCIS Oncotype score did not differ between the outcome groups in either cohort.

The 812 gene classifier likely represents several distinct biologic processes that promote recurrence and invasive progression. To further understand the biology and identify pathways involved in recurrence, we performed Gene Set Enrichment Analysis (GSEA) on DE genes between cases with 5-year recurrence vs the rest in TBCRC. We identified 11 Hallmark pathways significantly associated with early recurrence including those associated with proliferation, immune response, and metabolism.

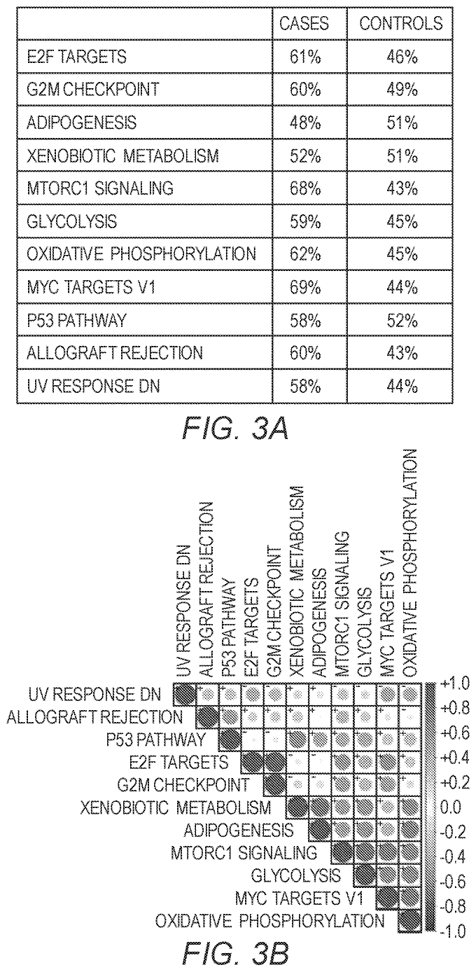

To further examine pathway activation status, we performed Gene Set Variation Analysis (GSVA) at the individual tumor level in 5-year outcome groups. Here, MYC and mTORc1 signaling were increased in cases vs controls and strongly correlated (FIGS. 3A-3B). We also observed high correlation between cell cycle linked G2M and E2F pathways. Further, Glycolysis and Oxidative Phosphorylation were increased in cases, and the significant positive correlation between these two pathways indicated that metabolically active tumors use both pathways. Overall, this analysis confirmed the finding from the differential abundance and GSEA analysis of 5-year outcome groups.

DCIS RNA Clustering Defines Expression Modules that Drive Outcome

Since proliferation and metabolism were identified as important pathways in recurrence, we next examined whether these pathways are driven by major DCIS phenotypes. Previous studies suggested that IBC subtypes do not fit well for DCIS. We hypothesized that a DCIS-specific classification scheme would better address DCIS biology. To investigate the biology behind the outcome analysis with emphasis on epithelial pathways, we performed unsupervised clustering of RNA-seq data from TBCRC (n=216) as well as an additional group of RAHBT cases (n=265) where we generated epithelial-enriched samples by laser capture microdissection (LCM) to evaluate tumor cell expression patterns without contributions from the tumor microenvironment.

We performed non-negative matrix factorization (NMF) on all protein coding genes (GENCODE v33) with non-zero variance, evaluated the fit of 2-10 clusters, and selected a 3-cluster solution based on silhouette width, cophenetic value, maximizing cluster number, and replication in RAHBT. The 3-cluster solution most reproducibly captured the biologic subgroups in both cohorts. To ensure the identified clusters were not an artifact of the clustering method, we ran consensus clustering in TBCRC, which rediscovered three clusters with high concordance with the NMF clusters (85.6%). In both cohorts, cluster 1 had significantly higher ERBB2 and lower ESR1 expression compared to clusters 2 and 3, which both had increased ESR1 expression. We termed the three clusters ER_{low}, quiescent, and ER_{high} respectively. To characterize these clusters, we conducted differential abundance analysis comparing each cluster individually to the other two combined (one-vs-rest). The deregulated pathways in each cluster were highly concordant across both cohorts, further supporting three transcriptional patterns in DCIS that are driven by the tumor cell compartment (P_{ERlow}=2.33×10⁻²; P_{quiescent}=8.37×10⁻²; P_{ERhigh}=9.20×10⁻¹⁰; hypergeometric test).

While we observed a differential expression of the estrogen response in the ER_{high} cluster vs ER_{low} cluster, the most striking patterns involved pathways associated with DCIS recurrence. Pathways including MYC, mTOR signaling, and cell cycle pathways were enriched in ER_{low} and significantly depleted in the quiescent cluster. Moreover, the Allograft Rejection, p53 and Adipogenesis pathways were high in ER_{low} and low in ER_{high}. Finally, ER_{high} tumors were depleted for UV Response Down and enriched for Oxidative Phosphorylation pathways, both of which were associated with recurrence. None of the recurrence-associated pathways were enriched in the quiescent cluster. The presence of the Allograft Rejection pathway in RAHBT LCM epithelial samples, though not significant, suggests that immune cells have infiltrated the epithelial compartment in the involved samples. Thus, the 3-cluster solution identified pathways associated with recurrence.

Genomic and transcriptomic-based classifications of IBC have characterized the spectrum of invasive breast cancer subtypes, but it remains unclear whether these accurately describe the spectrum of DCIS. To investigate, we applied the PAM50 classification to TBCRC and RAHBT LCM epithelial DCIS samples and evaluated the correlation of each sample to the centroid of its assigned subtype. We compared this correlation to IBCs from TCGA through repeated downsampling of the TCGA. The median correlation was consistently lower in DCIS compared to IBC, with the most pronounced difference in the basal-like subtype, as previously shown. Significantly decreased correlation was also observed for luminal A (P=3.13×10⁻³) and normal-like subtypes (P=6.21×10⁻³). UMAP projection of the DCIS transcriptome revealed clear deviations from the PAM50 centroids, and PAM50 failed to predict DCIS recurrence. These data suggest that while established IBC subtypes can be identified in DCIS, they do not fit DCIS as robustly as IBC, and are not prognostic in these premalignant lesions.

In support of the 3-cluster solution, we investigated MIBI protein expression for a subset of patients (n=71). The frequency of ER+ tumor cells was significantly higher in the quiescent and ER_{high} subtypes compared to ER_{low}(log₂ FC=2.73; P=2.11×10⁻⁵; Wilcoxon rank sum test) while HER2+ tumor cells were significantly higher in the ER_{low} subtype (log₂ FC=4.88; P=3.74×10⁻²; Wilcoxon rank sum test). Overall, the frequencies of ER+ and HER2+ tumor cells were well correlated with RNA abundance of ESR1 and ERBB2, respectively. PGR levels were upregulated in quiescent and ER_{high} compared to ER_{low}. Based on MIBI data, quiescent lesions were depleted for Ki67 (log₂ FC=−1.46; P=8.08×10⁻²; Wilcoxon rank sum test) and GLUT1 (log₂ FC=−2.64; P=8.47×10⁻³) positive tumor cells, vs ER_{high} and ER_{low} tumors, suggesting quiescent lesions are less proliferative and less metabolically active.

In their analysis of DCIS tumors and TME by MIBI, Risom et al. (Cell 185, 299-310.e18 (2022) identified myoepithelial E-cadherin expression as the most discriminative feature for risk of progression. To investigate this in relation to the identified RNA clusters, we compared the distribution of myoepithelial E-cadherin frequency by MIBI in matched RAHBT LCM RNA samples. We found that ER_{high} lesions had significantly higher myoepithelial E-cadherin frequency compared to ER_{low} and quiescent lesions (P≤0.026). While most recurrence-associated pathways were enriched in ER_{low} lesions, this points to a feature associated with recurrence amongst ER+ DCIS tumors, and highlights that there are multiple paths to progression in DCIS.

### Amplifications Characteristic of High-Risk of Relapse IBC Occur in DCIS

Next, we investigated how CNAs in DCIS contribute to pathways associated with DCIS recurrence. Amongst the 29 recurrent CNAs identified across both cohorts, we found 13 gains and 16 losses, occurring in 10.1-52.6% of DCIS samples (FDR<0.05; GISTIC2). The identification of these common CNAs was not biased by depth of sequencing, but two were associated with cohort (1p21.3 and 10p15.3 deletions). The most frequent alterations were gains of chromosomes 1q and 17q, including 17q12 where the ERBB2 oncogene is located, and loss of chromosome 17p, 16q, and 11q, confirming prior findings and notably reflecting the CNA landscape of IBC.

Next, we investigated if the distribution of Proportion of the Genome copy number Altered (PGA) was biased in the 5-year outcome groups or 812 gene classifier risk groups, but found no significant differential distribution. PGA was not correlated to sequencing depth, nor predictive of iBEs.

Early patterns of alterations may provide insight into the mechanisms of neoplastic lesion development and progression. To identify genomic subtypes in DCIS, we employed unsupervised NMF clustering of CNA segments on TBCRC and RAHBT jointly and identified eight clusters ranging in size from 2-98 samples which were not biased by depth of sequencing. CNA cluster 1 was characterized by chr20q13.2 amplification. Three clusters were characterized by chr17q amplification (Cluster 2: 17q11, Cluster 3: chr17q23.1, Cluster 4: chr17q12). Cluster 5 was had chr8p11.23 amplification, Cluster 6 chr11q13.3 amplification, and Cluster 7 amplification of MYC on chr8q24. Cluster 8, the largest group (n=98), represented a CNA quiet subgroup, characterized by the absence or diminished signal of these CNAs.

Integrative subgroups (ICs) is an IBC classification scheme based on genomic copy number and expression profiles. Intriguingly, despite the eight CNA clusters not being associated with recurrence several of these clusters were attributed to the presence or absence of CNAs characteristic of IC subtypes, namely the four high-risk of relapse ER+/HER2− subgroups (IC1,2,6,9) and the HER2-amplified (IC5) subgroup. Of note, these four high-risk integrative subgroups (IC1,2,6,9) account for 25% of ER+/HER2− IBC and the majority of distant relapses. Integrative subtypes are prognostic in IBC and improve the prediction of late relapse relative to clinical covariates. Understanding the clinical course of DCIS lesions harboring these high-risk invasive features is highly relevant in refining clinically meaningful risk associated with DCIS progression.

To identify enriched pathways in the eight CNA clusters, we investigated the differential abundance in matched RNA samples (DESeq2 one-vs-rest) and performed GSEA Hallmark analysis on the resulting gene lists. Clusters 6 (chr11q13 amplification) and 7 (chr8q24 (MYC) amplification) were enriched for pathways associated with recurrence (Allograft Rejection and Oxidative Phosphorylation, respectively), whereas Cluster 8 (CNA quiet) was depleted of recurrence associated pathways (Cell Cycle and mTORc1 signaling), and Cluster 6 was depleted of MYC targets. The remaining CNA clusters had no significant pathway enrichments. Thus, we identified a CNA-based cluster solution characterized by amplifications seen in high-risk IBC subtypes, including 17q12 (ERBB2) and 8q24 (MYC) amplification, some of which were significantly enriched or depleted for pathways associated with recurrence.

### The DCIS TME Reflects Distinct Immune and Fibroblast States

The Hallmark pathways identified represent a diverse set of biologic events and may involve different components of the DCIS ecosystem including the cells within the TME. Accumulating evidence has shown that the TME is crucial for cancer development and progression. To analyze the DCIS TME, we generated RAHBT LCM stromal samples by dissecting stromal tissue from the DCIS edge.

To identify the contribution of epithelial and stromal components to the 812 gene classifier, we performed differential abundance analysis between stromal (n=196) and epithelial (n=265) samples from the RAHBT LCM cohort. We identified 9748 DE genes (FDR<0.05) between epithelium and stroma (5161 epithelial, 4587 stromal). An analysis of the 812 classifier genes showed that 20% were expressed primarily in stromal/TME cells, and 34% in epithelium.

The MIBI method provides an orthogonal view of the TME and generates protein expression and identity of 16 different cell types including epithelial, fibroblasts, and immune cell types. We used adjacent TMA sections to analyze RNA and MIBI expression on the same ducts. We compared MIBI-based cell type distribution across samples with the inferred cell type distribution from RNA expression data using CIBERSORTx (CSx), allowing us to cross-validate findings and extend observations on cell composition to DCIS samples without MIBI data, including the TBCRC cohort.

To define discrete TME phenotypes, we performed shared nearest neighborhood clustering of stromal RNA data and identified four distinct DCIS-associated stromal clusters and DE genes (DESeq2 each-vs-rest). Pathway analyses, MIBI protein expression and cell type distribution, and CSx-inferred cell type distribution were used to describe major characteristics of each cluster, which were termed Immune dense, Desmoplastic, Collagen-rich, and Normal-like. There was a strong correlation with fibroblast states and immune cell density.

The Immune stromal cluster was the most distinct stromal subtype, with enrichment for the outcome-associated Allograft Rejection- and other immune activation pathways. MIBI and CSx data demonstrated a total abundance of immune cells more than twice that of any other cluster, with predominance of lymphoid over myeloid cells. A subgroup within this cluster was highly enriched for B cells, whereas another displayed overall balanced immune cell type composition. The Immune cluster also showed association with MIBI-identified T-cell and B-cell enriched neighborhoods, myoepithelial- and myeloid-enriched neighborhoods, and was enriched for the ER_{low} subtype.

The normal-like cluster was enriched for Gene Ontology pathways involved with ECM organization, Complement and Coagulation Cascades, Focal Adhesion, and PI3K-AKT signaling. The collagen-rich cluster was characterized by Collagen Metabolism, TGFb signaling, and Proteoglycans in Cancer, and Cell-Substrate and Focal Adhesion. This cluster had the highest fibroblast abundance and total myeloid cells, mostly associated with macrophages and myeloid dendritic cells (mDC). According to MIBI, this cluster was enriched in collagen and fibroblast associated protein positive (FAP+, VIM+, SMA+) myofibroblasts. The desmoplastic cluster was characterized by mammary gland development and fatty acid metabolism, high presence of VIM+, SMA+ myofibroblasts by MIBI, and higher levels of CD8+ T cells assessed by CSx vs the normal-like and collagen-rich clusters.

These analyses indicate that the immune response is present in a discrete subset of cases. However, outcome analysis by stromal subtype demonstrated a modest outcome difference, without major contribution from the Immune subcluster (P=0.12, log-rank test). We hypothesized that the outcome differences could be attributed to a subset of immune cells rather than the entire immune response, and analyzed CSx-inferred cell type distribution in 5-year outcome groups in TBCRC and RAHBT combined. We identified significantly higher levels of CD4+ T cells, myeloid- and plasmacytoid dendritic cells (pDC), monocytes, macrophages, and overall immune cells in cases vs. controls. Furthermore, we found that several cell types, including CD4 T-cells, mDCs, and pDCs, were significant predictors of any iBE 5 years after treatment (univariable Cox regression analysis). These differences in outcome groups were overall mirrored by CSx-inferred cell type distributions in the high- and low risk classifier groups. Finally, we investigated the distribution of CSx-based cell types in 5-year outcome groups stratified by iBE type. The results overall reflected the analysis in cases vs. controls, with the largest differences observed between invasive iBEs and controls.

Taken together, these results support the contributions of individual immune cells with high-risk outcomes. However, non-immune cell phenotypes are not well defined by this CSx approach but can still be identified as a biologic response. The desmoplastic cluster had the clearest and most favorable outcome (HR=0.23, P=0.06), despite being enriched for several recurrence-associated pathways, including proliferative signals (MYC and G2M checkpoint) associated with poor outcome in the epithelial compartment. This highlights the complexity and differential contribution from the stromal and epithelial compartments.

### Discussion

The aims of the HTAN Breast Pre-Cancer Atlas are to 1) develop a resource of multi-modal spatially resolved data from breast pre-invasive samples that will facilitate discoveries by the scientific community regarding the natural history of DCIS and predictors of progression to life-threatening IBC; and 2) populate that platform with data from retrospective cohorts of patients with DCIS and demonstrate its use to construct an atlas to test novel biologic insights. Here, we examined two well-annotated, retrospective, longitudinal patient cohorts with or without a subsequent iBE. The two cohorts have important and distinct differences. They comprise subjects from diverse geographical sites, race/ethnicities, median years of diagnosis, and time to recurrence. There were no significant differences in age at diagnosis or treatment across cohorts. Together, these cohorts comprise a large series of matched case-control samples allowing great statistical power to perform the comprehensive studies reported here. A particular strength of the study is the complementary nature of the two cohorts, allowing for validation of our findings, as well as the capability to separately study the epithelial and stromal components in RAHBT LCM samples. Future observations on a DCIS cohort undergoing watchful waiting would provide outcome results that may be more aligned with emerging personalized treatment strategies of DCIS, which could include non-surgical options.

DCIS is a heterogeneous disease with variable prognosis but has defied attempts to identify molecular factors associated with future progression. Previous studies have evaluated the prognostic value of biomarkers associated with outcomes, with conflicting conclusions for virtually all markers tested, including ER, HER2, immune markers such as tumor infiltrating lymphocytes, and stromal characteristics. Many promising leads have not been reproducible due to multiple factors, including lack of endpoint standardization, differences between cohorts, small sample size, and limited datasets for validation with long-term outcomes.

Herein, we have developed and validated an 812 gene classifier which independently predicted risk of both overall recurrence and invasive progression. This classifier was highly associated with outcome in a multivariable model which included treatment, age, grade, and clinical ER status; the classifier had a HR of 22.5 (95% CI 8.5-59.4) in the training set and 7.3 (95% CI 1.6-34.2) in the validation set, over four-fold higher than has been previously reported for other prognostic markers for DCIS.

Importantly, we found that this classifier was a stronger predictor of 5-year recurrence or progression than previously described clinical factors, including age at diagnosis, tumor grade, ER status, or treatment. The large dataset, with a high number of events, permitted an agnostic analysis of all genome-wide features and was thus less opportunistic than other, more limited studies. Further, since no a priori assumptions were made regarding whether to incorporate the molecular features of invasive cancer, we were able to construct a less biased predictor.

Our classifier is characterized by several Hallmark pathways including some related to cell cycle progression and growth factor signaling (E2F targets, G2M checkpoint, MYC targets, mTORc1 signaling) and metabolism (Glycolysis, Oxidative Phosphorylation). Examination of pathway activation status at the individual tumor level revealed the underlying complexity of the classifier. High correlation between cell cycle linked E2F and G2M pathways are consistent with a proliferation related signature. However, the strongest features of the classifier (distinguishing cases from controls) were MYC and MTORC1 signaling which are strongly correlated with each other but less so with the canonical proliferation pathways indicating that proliferation alone is not the central predictor. Interestingly, both Glycolysis and Oxidative Phosphorylation were increased in cases suggesting that heightened metabolic activity is associated with risk of progression regardless of whether it is anaerobic. Finally, Allograft Rejection, a broad immune pathway, was elevated in cases and in general appeared to be an independent component of the classifier. Overall, there are multiple components to this classifier that are elevated in different subsets of the tumors lending additional evidence that simplified predictors fail to capture the heterogeneity of the disease.

IBC has been genomically profiled with several approaches, including the PAM50 and IC classification schemes. While DCIS and IBC are part of the same neoplastic process, there are differences in the TME, evolutionary age, and inter-observer variability in diagnostic labeling at different stages of progression. This suggests that a DCIS-specific classification scheme would correlate better with biologic and clinical features of DCIS. Our analysis indicated the PAM50 subtypes are not apt for DCIS characterization, as previously described (Berghoitz et al., NPJ Breast Cancer 6, 26 (2020)). Instead, we identified three transcriptomic DCIS subgroups, characterized by ER signaling, proliferation and metabolism. These subtypes more accurately capture the spectrum of DCIS biology than IBC-derived subtypes, and represent the fundamental genomic organization at this early stage of breast neoplasia. They may represent the earliest variation in neoplasia transcriptome, potentially applicable to earlier stages such as hyperplasias.

There are several possible reasons why traditional IBC classifiers do not perform well on DCIS. HER2 expression is more common at the DCIS stage than at the IBC stage, which may lead to a different transcriptomic distribution in DCIS vs IBC. Many ER-DCIS express HER2 without amplification, in contrast to IBC, where the HER2-amplified subtype is clearer. Moreover, DCIS cells are confined to the epithelial compartment and interact with myoepithelial cells and the basement membrane, thus presumably restricted by rules of differentiation that govern normal epithelial cells, which could constrain the transcriptomic variability of neoplastic cells and in turn possible subtypes. Finally, the evolutionary age of the neoplasm may influence classification differences in DCIS vs IBC. By comparing WGS data from DCIS and IBCs, we found that the same constellation of copy number changes was present in both, consistent with previous studies. While DCIS had fewer genomic alterations than IBC, and a larger group of DCIS was classified as genomically quiescent, recurrent genomic events that drive the IBC-based IC scheme were evident at the DCIS stage.

A unique aspect of our study is the separate profiling of stromal and epithelial components through CSx analysis of LCM-derived RNA coupled with in situ MIBI protein expression. We identified four stromal subtypes characterized by distinct pathways, stromal-, and immune cell composition. Specific stromal patterns were correlated with epithelial expression patterns, and particularly HER2+/ER− DCIS were associated with a stronger immune response, potentially associated with co-amplification of ERBB2 (HER2) and chemokine encoding genes on the 17q12 chromosomal region. A limitation of this study is that our CSx approach did not facilitate identification of non-immune stromal cell types.

Generating a DCIS atlas is similar to the effort of TCGA for IBC, but there are important differences. Working with DCIS samples is considerably more challenging; while IBC tumors are evident by gross exam, and can be easily obtained as fresh, fresh frozen, or archival material, this is not the case for pre-invasive lesions. DCIS can sometimes be recognized radiographically but is only precisely detailed by pathologic examination, making prospective tissue collection a challenge. Moreover, the transition from intraepithelial to invasive neoplasia is definitional for IBC. For DCIS, such a clear-cut definition does not exist. DCIS is broadly defined by cytologic and architectural changes compared to normal breast tissue by a growth of neoplastic cells in the inter-epithelial compartment.

One issue that should be noted is the genetic relationship between the primary DCIS and the subsequent ipsilateral cancer. Recent work on a large cohort indicates that 18% of ipsilateral invasive events may be unrelated to the primary DCIS based on mutations and CNAs. Non-clonal recurrences were more likely to be in a different breast quadrant and have discordant ER expression whereas time to recurrence and patient age were not significantly associated with clonality. While we did not examine the recurrences in the current study to determine clonality, it is likely that a similar fraction would be identified as “unrelated.” We anticipate that further refinement and validation of our classifier will be strengthened by eliminating non-clonal iBEs.

In conclusion, we have developed a genomic classifier that predicts both recurrence and invasive progression, using large, comprehensively annotated case-control data sets of primary DCIS. The classifier is comprised of both epithelial and stromal features. Our findings support that progression is a process that requires both invasive propensity among the DCIS cells and stromal permissiveness in the TME. We propose this classifier as the basis for a future clinical test to assess outcomes in patients with primary DCIS to guide a more individualized therapy, based on biologic risk. Future work will include further validation of the classifier and translation to clinical implementation.

### Experimental Model and Subject Details

### Cohort Collection and Sample Acquisition

### RAHBT Cohort

The Resource of Archival Breast Tissue (RAHBT) is a data/tissue resource established by Drs. Allred and Colditz in 2008 focused on premalignant or benign breast disease. Uniform coding of premalignant lesions assures greater consistency and use of research. Follow-up through hospital record linkages documents subsequent breast lesions including IBC. The entire study population includes women ages 18 and older with documented cases of premalignant breast disease (including carcinoma in situ). The study was approved by the Washington University in St. Louis Institutional Review Board (IRB ID #: 201707090).

Women were identified as eligible through seven primary sources: Washington University School of Medicine Departmental databases (Surgery, Radiation Oncology, Pathology, and Radiology), and the Siteman Oncology Services Database (local tumor registry), the St. Louis Breast Tissue Repository, and the Women's Health Repository. We reviewed all records, excluded women with cancer prior to qualifying premalignant lesions and identified 1831 unique women with DCIS or DCIS and subsequent recurrence. A common data set with pathologic details, risk factor data, treatment, and unique identifiers was created and used to follow these women for subsequent breast lesions. Centralized pathology review confirmed 174 cases of DCIS with recurrent lesions. For each case (with subsequent ipsilateral or contralateral breast events) we matched two controls who remained free from subsequent breast events based on race, year of diagnosis (+/−5 years), age at diagnosis (+/−5 years), and type of definitive surgery (mastectomy or lumpectomy). For each DCIS diagnosis we retrieved slides and blocks for pathology review, secured a whole slide image of each sample, marked for TMA cores, and prepared for laboratory processing. A total of 172 cases and 338 controls were cored for TMAs. Breast pathology review was completed by Drs. Allred, Warrick, DeSchryver, and Veis.

To define an external validation data set that used identical eligibility criteria to TBCR 038 including year of initial DCIS diagnosis, we identified an additional set of cases from RAHBT and used comparable laboratory procedures for RNA-seq.

For RAHBT, 97 patients were analyzed by RNA-seq (Table 2). The median age at diagnosis was 53, and median year of diagnosis 2006. Time to recurrence with ipsilateral IBC was 36 months, and to diagnosis of ipsilateral DCIS 46.9 months. For women in the cohort with no iBEs, median follow-up extended to 141 months. The total number of deaths by any cause was six. Treatment of initial DCIS ranged from lumpectomy with radiation (66.0%), and no radiation (10.3%) and mastectomy (23.7%). This subset of the RAHBT cohort was composed of 35.1% African American women.

For RAHBT LCM, 265 patients were analyzed by RNA-seq. The median age at diagnosis was 53, and median year of diagnosis 2002. Time to recurrence with ipsilateral IBC was 80 months, and to diagnosis of ipsilateral DCIS 50 months. For women in the cohort with no iBEs, median follow-up extended to 111 months. Treatment of initial DCIS ranged from lumpectomy with radiation (52%), and no radiation (18%) and mastectomy (28%). This subset of the RAHBT cohort was composed of 25% African American women.

### TBCRC 038 Cohort

TBCRC 038 is a retrospective multi-center study activated at 12 participating TBCRC (Translational Breast Cancer Consortium) sites, which identified women treated for ductal carcinoma in situ (DCIS) at one of the enrolling institutions between Jan. 1, 1998 and Feb. 29, 2016. The TBCRC and the Department of Defense (DOD) approved this study for the collection of archival tissues. Duke served as the initiating and central site for all data, samples, assays, and analysis. The study was approved by the Duke Health Institutional Review Board (Protocol ID: Pro00068646) as well as the IRB at each participating institution. Individual sites reviewed medical records to identify patients eligible for the study.

Study eligibility criteria included: Women aged 40-75 years at diagnosis of DCIS without invasion; no prior treatment for breast cancer; and definitive surgical excision with no ink on tumor margins and treated with mastectomy, lumpectomy with radiation, or lumpectomy. Cases (patients with subsequent iBEs) were matched 1:1 to controls with at least 5 years of follow-up without subsequent iBEs. Matching was based on year of diagnosis (+/−5 years), age at diagnosis (+/−5 years), and DCIS nuclear grade (high grade vs. non-high grade). All cases consisted of initial diagnosis of pure DCIS, with ipsilateral recurrence occurring no less than 12 months from date of primary diagnosis. Clinical data, including treatment data, were collected at each site, and standardized data points were entered into a web-based portal. Tumor tissue was collected from FFPE blocks and cut into Sum sections. All slides were scanned and reviewed centrally by a breast pathologist (AH) to confirm the diagnosis. Tumor tissue marked by the pathologist was macrodissected for bulk analysis assays.

The 216 patients from the TBCRC cohort analyzed by RNA-seq (Table 2) includes 95 women without iBE after 5 or more years, 66 with DCIS iBEs, and 55 with IBC iBEs. Median time to IBC iBE for this subset was 58 months and 40 months to DCIS iBE. The total number of deaths by any cause was 12.30% of this subset were African American.

### Method Details

### TMA Construction

Qualified DCIS or subsequent lesion slides were assembled for pathology review. The research breast pathologist marked the slides for best area to core (1 mm) for the carcinoma in situ and later event. The TMAs were designed such that cases/controls were assigned randomly on the map. The Beecher Tissue Arrayer was used to take a core from the patient donor block and place it in the designated area of the recipient TMA block. Slides were then cut for research purposes, and stained H&E and unstained slides were prepared. The TMAs were stored in the St. Louis Breast Tissue Registry Lab at room temperature.

### Slide Cutting

A TMA cutting breakdown was established to include slides for laser capture microdissection (LCM PEN membrane glass slides) sequencing, multiplex protein (MIBI high-purity gold-coated slides) staining and charged glass slides for FISH analysis of the RAHBT TMAs. The order of the slides for the different assays was as follows:
Slide 1-3: FISH/routine IHC—4 um slices on charged slides
Slide 4-6: RNA/DNA sequencing—7 um slices on LCM membrane glass slides
Slide 7: MIBI analysis—4 um slices on gold coated slides
Slide 8-10: FISH/routine IHC—4 um slices on charged slides
Slide 11-13: RNA/DNA sequencing—7 um slices on LCM membrane slides
Slide 14: MIBI analysis—4 um slices on gold coated slides
Slide 15-17: FISH/routine IHC—4 um slices on charged slides
Slide 18 H&E stained.

### Digital H&E Generation (Scanners)

At Washington University School of Medicine, the H&E original slide and TMA slide for RAHBT was imaged (20×) by Aperio AT2 (Leica). ImageScope provides the software for viewing the slides. Images are stored on secure servers in the Dept of Pathology, Washington University School of Medicine.

### Pathologic Analysis and Masking

For the TBCRC cohort, whole slide images of the H&E slide made from the block sourced for DNA and RNA was reviewed and scored for grade, presence of necrosis and architecture by a breast pathologist. For the RAHBT LCM cohort, H&E images from the TMAs were used to score for grade, presence of necrosis and architecture by four breast pathologists. Areas of DCIS and normal tissue from the RAHBT TMAs were annotated and masked for LCM by two breast pathologists.

### Laser Capture Microdissection

Consecutive sections of tissue microarray blocks were cut and mounted on PEN membrane slides. Slides were dissected immediately after staining on an Arcturus XT LCM System based on the masked areas. Epithelial and stromal sections were dissected separately. Each sample adhere to a CapSure HS LCM Cap (Thermo Fisher #LCM0215). After LCM, the cap was sealed in an 0.5 mL tube (Thermo Fisher #N8010611) and stored at −80° C. until library preparation. The matching epithelial regions in consecutive slides were dissected for corresponding DNA libraries.

### RNA-Sequencing (Smart-3Seq)

Sequencing libraries were prepared according to the Smart-3SEQ method starting from dissected FFPE tissue on an Arcturus LCM HS Cap, except for the unique P5 index and universal P7 primers. Three control samples were added to each library preparation batch and sequence batch to allow batch effect analysis. Libraries were pooled together according to qPCR measurements and prepared according to the manufacturer's instructions with a 1% spike-in of the PhiX control library (Illumina #FC-110-3002) and sequenced on an Illumina NextSeq 500 instrument with a High Output v2.5 reagent kit (Illumina #20024906).

### ER, HER2 Status

Clinical ER status (by IHC) was available for 83.3% (180 of 216) of the TBCRC cohort, 83.5% (81 of 97) of the RAHBT cohort, and 46.8% (124 of 265) of the RAHBT LCM cohort.

Additionally, we called ER and HER2 positivity based on mRNA abundance levels of ESR1 and ERBB2, respectively. We applied a Gaussian mixture model with two components using the mclust R package (v5.4.7).

### PAM50 and IC10

PAM50 subtypes were called using the genefu v2.22.1 R package. We compared the PAM50 subtypes called by genefu against subtypes called adjusting for the expected proportion of ER+ samples, as implemented in. We found both methods to be highly concordant (>96% concordance). We compared the correlation of DCIS and IBC samples to the PAM50 centroids within the genefu R package using Spearman's correlation. We also compared the silhouette widths based on Euclidean distances of the PAM50 subtypes to the de novo DCIS subtypes using the cluster R package (v2.1.1). IC10 subtypes were called using the iC10 (v1.5) R package. PAM50 subtypes were called in TBCRC and RAHBT separately, using the same protocols, given the differences in measurement techniques used in the two cohorts.

To compare PAM50 centroids in DCIS to TCGA: The TCGA cohort was downsampled to match the size of the DCIS cohort. The downsampling was repeated 1,000 times, and the median correlation for each of the 1,000 iterations was compared to the median DCIS correlations.

### Differential Abundance Analyses

Differential abundance analysis was performed using the R package DESeq2 v1.30.1 with default options. P-values were adjusted for multiple testing using the Benjamini-Hochberg method. FDR<0.05 was considered significant for all DESeq2 analyses. Reads matrices were VST normalized for downstream analyses.

### Unsupervised Clustering: Non-Negative Matrix Factorization

We identified RNA and CNA based clusters by non-negative matrix factorization using the NMF R package v0.23.0. Each NMF rank was run 30 times to evaluate cluster stability. We comprehensively evaluated 2-10 clusters for each data type and evaluated cluster fit by cophenetic and silhouette values. RNA clusters were first discovered in TBCRC and replicated in RAHBT. We evaluated replication by quantifying the concordance of de novo clusters identified in RAHBT vs clusters determined from centroids identified in TBCRC.

CNA clusters were discovered in TBCRC and RAHBT jointly and compared against clusters identified in TBCRC and RAHBT individually to ensure robustness.

### CIBERSORTx

Using single-cell RNA-seq datasets, a breast specific signature matrix was built to resolve proportions of tumor, fibroblasts, endothelial and immune cells from bulk RNA-seq data. scRNAseq data was downloaded from Gene Expression Omnibus database (GEO data repository accession numbers GSE114727, GSE114725). Normalized counts were obtained using Seurat R package (v3.2.0), and used as single cell matrix input alongside with their cell type identities (code available: cibersortx.stanford.edu/, default parameters for “Create Signature Matrix/scRNAseq input data”). The resultant signature matrix contained 3484 genes and allowed to resolve different immune cell types, including B, CD8 T, CD4 T, NKT, NK, mast cells, neutrophils, monocytes, macrophages and dendritic cells, “Impute Cell Fractions/Enable batch correction S-mode”, and default parameters). The signature matrix was first in-silico validated. In order to test the accuracy of the signature matrix, a set of samples ( 1/10 of each type) from the same scRNAseq dataset was reserved to build a synthetic matrix of bulk RNA-seq data. By mixing different proportions of single cell transcripts, the synthetic bulk was used to predict cell type proportions and subsequently correlated with the true proportions used to build the synthetic mix. Pearson's coefficient was >0.75 in all the cases, and most >0.9. The aforementioned matrix was used to deconvolve the LCM RNA-seq samples and to compare CSx-estimated cell abundance with MIBI-identified cell types. Cell abundance between groups was compared by Wilcoxon rank sum test followed by Benjamini-Hochberg correction for multiple testing.

### Shared Nearest Neighbor Clustering

LCM stromal samples from RAHBT were classified using the Shared Nearest Neighbor clustering method implemented in the Seurat R package (v3.2.0). Data was normalized by negative binomial regression (sctransform R package, v0.3.2, variable.feature.n=“all.genes”). The first 15 principal components were used to identify the clusters and 16 different resolutions were compared, selecting resolution 0.75 and four clusters as the final solution. Positive markers were selected at a minimum fraction of 0.25 and the resultant gene list was used to further characterize each cluster by gene ontology and KEGG pathway analysis, implemented in clusterProfiler R package (version 3.18.1).

### Pathway & Gene Set Enrichment Analyses

Gene set enrichment analyses were performed using fgsea R package (v1.12.0) based on the MSigDB Hallmark pathways v7.4. All genes from differential abundance analyses were included and were ranked by their signed adjusted P-values. Pathways were considered enriched if adjusted P-values<0.05. We evaluated pathway concordance across the DCIS subtypes using a hypergeometric test.

Single sample gene set variation analysis was performed using the GSVA R package (v1.38.2) using default parameters.

### Outcome Analysis

Associations with time to event were quantified using Cox Proportional Hazard model correcting for treatment as indicated in the text. To standardize follow-up across TBCRC and RAHBT, we censored the follow-up time at 250 months, the maximum follow-up time in TBCRC. Kaplan-Meier plots as implemented in the R packages survival (v3.2.10) and survminer (v0.4.9) were used to visualize outcome differences.

The 812 gene classifier was built using the cforest implementation of Random Forest in the Caret (v6.0-91) R package using default parameters. The TBRCR cohort was used as the training cohort and the model was tested on the RAHBT cohort. Hyperparameters were tuned on the training cohort using four-fold cross validation. The mtry parameters 5, 20, 50, 100, 200, 500, and 800 were tested and the optimal mtry selected was 5. Accuracy of the classifier was assessed using ROC curve, Precision, Recall, and F1 score.

Breast cancer data (BRCA) from TCGA was downloaded from www.cancer.gov/tcga. A total of 1064 samples with available follow-up information was used to test the 812 gene classifier towards progression-free survival and overall survival as defined in the TCGA-BRCA metadata.

RNA for the TCGA samples was normalized using the same protocols as the DCIS RNA-sequencing (TBCRC and RAHBT cohorts, above). The accuracy of the classifier in the TCGA cohort was assessed using ROC curve, Precision, Recall, and F1 score.

### DNA-Sequencing

Genomic DNA was isolated from LCM FFPE cells using PicoPure DNA Extraction kit (Thermo Fisher Scientific #KIT0103). 50 ul lysis buffer with Proteinase K were added to each sample and incubated at 65° C. overnight. After inactivating proteinase K, the genomic DNA was cleaned up with AMPure XP beads at 3:1 ratio (Beckman Coulter #A63880) and eluted in the 10 mM Tris-HCl (pH8.0).

DNA Libraries were constructed with KAPA HyperPlus Kit (Kapa Biosystems #07962428001). Barcode adapters were used for multiplexed sequencing of libraries with SeqCap Adapter Kit A (Kapa Biosystems #7141530001). DNA libraries were amplified by 19 PCR cycles. AMPure XP beads were used for the size selection and cleaning up. DNA libraries were eluted in the 30 μL 10 mM Tris-HCl (pH8.0).

Library size distribution was assessed on an Agilent 2100 Bioanalyzer using the DNA 1000 assay and the concentration was measured by Qubit® dsDNA HS Assay Kit (Thermo Fisher Scientific #Q32851). For each lane, 12 samples were pooled and sequenced by Novogene (Sacramento, CA, US) on the Illumina HiSeq Platform, collecting 110 G per 275M reads output of paired-end reads of 150 bp length.

### Identification of Recurrent CNAs (GISTIC)

Recurrent CNAs were identified from purity-adjusted segment CNA calls from QDNASeq for 228 DCIS samples using GISTIC2 v2.0.23 run with the following parameters: -ta 0.3 -td 0.3 -qvt 0.05 -brlen 0.98 -conf 0.95 -armpeel 1 -res 0.01 -rx 0. To ensure CNAs were not biased by sequencing depth, recurrent CNAs significantly associated (FDR<0.05) with the number of uniquely mapped reads were filtered out. Associations were quantified by Mann-Whitney test. The number of uniquely mapped reads was determined from samtools flagstat (v1.9).

### MIBI

We used a MIBI panel consisting of 37 metal-conjugated antibodies that capture 16 different cell types including epithelial, fibroblasts, and immune cell types. We took tissue sections from adjacent sections to those used for RNA-seq to spatially align the same ducts for both MIBI and RNA. For full details of the MIBI methods, see the companion paper. Briefly, antibodies were conjugated to isotopic metal reporters. Tissues were sectioned (5 μm section thickness) from tissue blocks on gold and tantalum-sputtered microscope slides. Imaging was performed using a MIBI-TOF instrument with a Hyperion ion source.

Multiplexed image sets were extracted, slide background-subtracted, denoised, and aggregate filtered. Nuclear segmentation was performed using an adapted version of the DeepCell CNN architecture. Single cell data was extracted for all cell objects and area normalized. The FlowSOM R package v1.22.0 was used to assign each cell to one of five major cell lineages (tumor, myoepithelial, fibroblast, endothelial, immune). Immune cells were subclustered to delineate B cells, CD4+ T cells, CD8+ T cells, monocytes, MonoDC cells, DC cells, macrophages, neutrophils, mast cells, double-negative CD4−CD8− T cells, and HLADR+ APC cells. Tumor and fibroblast cells were similarly sub clustered to reveal phenotypic subsets. A total of 16 cell populations were quantified and analyzed. For full details of the MIBI methods, see the companion paper.

### Data Visualization

Boxplots, heatmaps, scatterplots and barplots were generated using the BoutrosLab.plotting.general R package v6.0.3, or the R packages ggplot2 (v3.3.3, boxplots), corrplot (v0.84, scatterplots), and ComplexHeatmap (v.2.6.2, heatmaps). UMAPs were generated using the umap (v0.2.7.0) R package with the number of genes indicated in the text. Mosaic plots were generated using the vcd (v1.4.8) R package.

### Quantification and Statistical Analysis

### RNA-seq Processing

RNA sequencing data was processed with 3SEQtools. Single-end Illumina FASTQ files were generated from NextSeq BCL files with bcl2fastq (v2.20.0.422) and then aligned to reference hg38 with STAR aligner (v2.7.3a). Samples that did not meet a minimum threshold of uniquely aligned reads were filtered out. The samples in this study averaged 1.11 million uniquely aligned reads. Gene expression matrices of raw and normalized read counts were produced from BAM files with featureCounts (v1.6.4) of the Subread package (v2.4.2) and GENCODE Release 33.

Read counts were normalized using the variance stabilizing transformation (VST) implemented in the R package, DESeq2 (v1.30.1). The VST normalization procedure normalizes for library size and returns a matrix that is approximately homoscedastic. The same normalization method was used for both the TBCRC and RAHBT cohorts individually.

### DNA-Seq Processing

Low-pass WGS data were preprocessed using the Nextflow-base pipeline Sarek v2.6.1 with BWA v0.7.17 for sequence alignment to the reference genome GRCh38/hg38 and GATK v4.1.7.0 to mark duplicates and calibration. The recalibrated reads were further processed and filtered for mappability, GC content using the R/Bioconductor quantitative DNA-sequencing (QDNAseq) v1.22.0 with R v3.6.0. For QDNAseq, 50-kb bins were generated from (doi.org/10.5281/zenodo.4274556). We kept only autosomal sequences after filtering due to low-depth mappability and GC correction. We used the QDNAseq corrected output and segmented for CN analysis using the circular binary segmentation (CBS) algorithm from DNAcopy R/Bioconductor package v1.60.0. Copy number aberrations were called using CGHcall v2.48.0. The R/Bioconductor package ACE v1.4.0 was used to estimate purity and ploidy. Proportion of the genome copy number altered (PGA) was calculated based on CNAs with |log 2 ratio|>0.3 based on the following: PGA=(number of bases in CNA)/(total number of bases profiled)

### Statistical Analyses

We used Mann-Whitney U test to compare continuous distributions between two groups, as specified in the text. We used the Kruskal-Wallis test to compare continuous values between three groups. All statistical analyses were implemented in the R statistical language (v3.6.1). P-values were corrected for multiple hypothesis testing via Bonferroni (when <10 independent tests) or Benjamini & Hochberg (when >10 independent tests).

Further details are provided in Strand et al., Cancer Cell 40, 1-16 (2022), and its accompanying Supplementary Materials, which are incorporated by reference herein.

One skilled in the art will readily appreciate that the present disclosure is well adapted to carry out the objects and obtain the ends and advantages mentioned, as well as those inherent therein. The present disclosure described herein is representative of preferred embodiments, which are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The foregoing is illustrative of the present invention, and is not to be construed as limiting thereof. The invention is defined by the following claims, with equivalents of the claims to be included therein.

1. A method for processing a tissue sample (e.g., biopsy) from a subject, comprising:
(a) providing the sample from the subject, said sample comprising cells of a breast tissue site of interest, said site of interest comprising or suspected of comprising ductal carcinoma in situ (DCIS) (e.g., suspected based on an abnormal mammogram), wherein said cells comprise a plurality of messenger ribonucleic acid (mRNA) molecules; and
(b) optically detecting an expression level of said plurality of mRNA molecules to thereby quantify expression levels of a plurality of genes in the cells.

2. The method of claim 1, wherein (b) comprises reverse transcribing said plurality of mRNA molecules to generate a plurality of complementary deoxyribonucleic acid (cDNA) molecules, and subsequently optically detecting said plurality of cDNA molecules.

3. The method of claim 2, further comprising, prior to optically detecting, performing nucleic acid amplification of the plurality of cDNA molecules, and optionally wherein said nucleic acid amplification comprises polymerase chain reaction (PCR) or isothermal amplification.

4. (canceled)

5. The method of claim 2, wherein said optically detecting comprises detecting an optical signal from a probe coupled to a cDNA molecule of said plurality of cDNA molecules, and optionally wherein said optical signal is a fluorescent signal.

6. (canceled)

7. The method of claim 1, further comprising processing said cells to access (and optionally extract) the plurality of mRNA molecules prior to said optically detecting.

8. The method of claim 1, wherein said sample comprises a heterogeneous mixture of cells (e.g., mixed epithelial and stromal cells) (e.g., from a core biopsy or lumpectomy).

9. The method of claim 1, wherein the subject has undergone surgery for DCIS (i.e., lumpectomy).

10. The method of claim 1, wherein the subject has not undergone surgery for DCIS.

11. The method of claim 1, wherein said plurality of genes comprises at least 5, 10, 15, 20, 30, 40, 50, 60, 70, 80, 90 or 100 of the genes listed in Table 1, at least 30, 50, 80, 100, 200, or 300 of the genes listed in Table 1, or at least 100, 300, 500, 600, 700, or 800 of the genes listed in Table 1.

12.-13. (canceled)

14. The method of claim 1, further comprising determining an increased or decreased risk of recurrence and/or progression of DCIS based upon the expression levels of the plurality of genes.

15. The method of claim 14, further comprising treating the subject upon determining an increased risk of recurrence and/or progression of DCIS, wherein the treating comprises surgery, radiation, and/or chemotherapy (e.g., endocrine therapy).

16. (canceled)

17. A method for generating a classifier, comprising:
(a) providing tissue samples (e.g., biopsies) from a plurality of subjects, said samples comprising cells of a breast tissue site of interest, said site of interest comprising or suspected of comprising ductal carcinoma in situ (DCIS) (e.g., suspected based on an abnormal mammogram), wherein said cells comprise a plurality of messenger ribonucleic acid (mRNA) molecules;
(b) optically detecting an expression level of said plurality of mRNA molecules to thereby quantify expression levels of a plurality of genes in the cells; and
(c) using the expression levels of the plurality of genes to train a classifier, said classifier capable of determining a risk of DCIS recurrence and/or progression,
to thereby generate the classifier.

18. The method of claim 17, wherein (b) comprises reverse transcribing said plurality of mRNA molecules to generate a plurality of complementary deoxyribonucleic acid (cDNA) molecules, and subsequently optically detecting said plurality of cDNA molecules.

19. The method of claim 18, further comprising, prior to optically detecting, performing nucleic acid amplification of said plurality of cDNA molecules, optionally wherein said nucleic acid amplification comprises polymerase chain reaction (PCR) or isothermal amplification.

20. (canceled)

21. The method of claim 18, wherein said optically detecting comprises detecting an optical signal from a probe coupled to a cDNA molecule of said plurality of cDNA molecules, and optionally wherein said optical signal is a fluorescent signal.

22. (canceled)

23. The method of claim 17, further comprising processing said cells to extract the plurality of mRNA molecules prior to said optically detecting.

24. The method of claim 17, wherein said sample comprises a heterogeneous mixture of cells (e.g., mixed epithelial and stromal cells) (e.g., from a core biopsy or lumpectomy).

25. The method of claim 17, wherein the subject has undergone surgery for DCIS (i.e., lumpectomy).

26. The method of claim 17, wherein the subject has not undergone surgery for DCIS.

27. The method of claim 17, wherein the classifier is agnostic to the biological type of DCIS and/or subsequent invasive cancer.

28. The method of claim 17, wherein the classifier is trained based on a subsequent ipsilateral occurrence of DCIS and/or invasive breast cancer in the plurality of subjects (e.g., within about 3, 5 or 8 years from collection of the tissue samples).

29. A system for determining the risk of DCIS recurrence and/or progression in a subject in need thereof, comprising:
at least one processor;
a sample input circuit configured to receive a tissue sample from the subject;
a sample analysis circuit coupled to the at least one processor and configured to determine gene expression levels of the tissue sample;
an input/output circuit coupled to the at least one processor;
a storage circuit coupled to the at least one processor and configured to store data, parameters, and/or a classifier; and
a memory coupled to the processor and comprising computer readable program code embodied in the memory that when executed by the at least one processor causes the at least one processor to perform operations comprising:
controlling/performing measurement via the sample analysis circuit of gene expression levels of a plurality of genes in said tissue sample;
optionally, normalizing the gene expression levels to generate normalized gene expression values;
retrieving from the storage circuit a DCIS classifier;
entering the gene expression values into the classifier; and
determining a score or risk of DCIS recurrence and/or progression based upon said classifier.

30. The system of claim 29, wherein said plurality of genes comprises at least 5, 10, 15, 20, 30, 40, 50, 60, 70, 80, 90 or 100 of the genes listed in Table 1, wherein said plurality of genes comprises at least 30, 50, 80, 100, 200, or 300 of the genes listed in Table 1, or wherein said plurality of genes comprises at least 100, 300, 500, 600, 700, or 800 of the genes listed in Table 1.

31.-32. (canceled)

33. The system of claim 29, wherein the classifier was generated by a method comprising:
(a) providing tissue samples (e.g., biopsies) from a plurality of subjects, said samples comprising cells of a breast tissue site of interest, said site of interest comprising or suspected of comprising ductal carcinoma in situ (DCIS) (e.g., suspected based on an abnormal mammogram), wherein said cells comprise a plurality of messenger ribonucleic acid (mRNA) molecules;
(b) optically detecting an expression level of said plurality of mRNA molecules to thereby quantify expression levels of a plurality of genes in the cells; and
(c) using the expression levels of the plurality of genes to train a classifier, said classifier capable of determining a risk of DCIS recurrence and/or progression.
